(19) United States
(12) Patent Application Publication
(10) Pub. No.: US 2016/0289236 A1
(43) Pub. Date: Oct. 6, 2016

(54) Protein Kinase Inhibitors

(71) Applicant: PHARMASCIENCE INC., Montreal, Québec (CA)

(72) Inventors: Alain Laurent, Montreal, Québec (CA); Yannick Rose, Montreal, Québec (CA)

(73) Assignee: Pharmascience Inc., Montreal, Québec (CA)

(21) Appl. No.: 15/037,613

(22) PCT Filed: Nov. 19, 2014
(86) PCT No.: PCT/CA2014/000836

§ 371 Date: May 18, 2016

(30) Foreign Application Priority Data
Nov. 19, 2013 (CA) 2833701

(51) Int. Cl. C07D 487/04; A61K 45/06; A61K 31/519

(52) U.S. Cl. CPC C07D 487/04; A61K 31/519; A61K 45/06

(57) ABSTRACT

The present invention relates to a novel family of protein kinase inhibitors, more specifically the present invention is directed to inhibitors of the members of the Tec or Src protein kinase families. The present invention also relates to the processes of preparation of these compounds, their intermediates, to pharmaceutical compositions comprising them, and to their use in the treatment of proliferative, inflammatory, autoimmune, or infectious diseases, disorders, or conditions in which protein kinase activity is implicated.

### FIELD OF INVENTION

The present invention relates to a novel family of protein kinase inhibitors, to the processes for preparation of compounds and their intermediates, to pharmaceutical compositions comprising them, and to their use in the treatment of proliferative, inflammatory, infectious, or autoimmune diseases, disorders, or conditions in which protein kinase activity is implicated.

### BACKGROUND OF THE INVENTION

Protein kinases are a large group of intracellular and transmembrane signaling proteins in eukaryotic cells (Manning G. et al, (2002) Science, 298: 1912-1934). These enzymes are responsible for transfer of the terminal (gamma) phosphate from ATP to specific amino acid residues of target proteins. Phosphorylation of specific amino acid residues in target proteins can modulate their activity leading to profound changes in cellular signaling and metabolism. Protein kinases can be found in the cell membrane, cytosol and organelles such as the nucleus and are responsible for mediating multiple cellular functions including metabolism, cellular growth and differentiation, cellular signaling, modulation of immune responses, and cell death. Serine kinases specifically phosphorylate serine or threonine residues in target proteins. Similarly, tyrosine kinases, including tyrosine receptor kinases, phosphorylate tyrosine residues in target proteins. Tyrosine kinase families include: Tec, Src, Abl, Jak, Csk, Fak, Syk, Fer, Ack, and the receptor tyrosine kinase subfamilies including EGFR, FGFR, VEGFR, RET and Eph.

Kinases exert control on key biological processes related to health and disease. Furthermore, aberrant activation or excessive expression of various protein kinases are implicated in the mechanism of multiple diseases and disorders characterized by benign and malignant proliferation, as well as diseases resulting from inappropriate activation of the immune system (Kyttaris V. C., Drug Des. Devel. Ther., 2012, 6:245-50 and Fabbro D. et al. Methods Mol. Biol., 2012, 795:1-34). Thus, inhibitors of select kinases or kinase families are expected to be useful in the treatment of cancer, vascular disease, autoimmune diseases, or inflammatory conditions including, but not limited to: solid tumors, hematological malignancies, thrombus, arthritis, graft versus host disease, lupus erythematosus, psoriasis, colitis, illeitis, multiple sclerosis, uveitis, coronary artery vasculopathy, systemic sclerosis, atherosclerosis, asthma, transplant rejection, allergy, dermatomyositis, pemphigus, and the like.

Tec kinases are a family of non-receptor tyrosine kinases predominantly, but not exclusively, expressed in cells of hematopoietic origin (Bradshaw J. M. Cell Signal. 2010, 22:1175-84). The Tec family includes Tec, Bruton's tyrosine kinase (Btk), inducible T-cell kinase (Itk), resting lymphocyte kinase (Rlk/Txk), and bone marrow-expressed kinase (Bmx/Etk). Btk is important in B-cell receptor signaling and regulation of B-cell development and activation (W. N. Khan et al. Immunity, 1995, 3:283-299 and Satterthwaite A. B. et al. Immunol. Rev. 2000, 175: 120-127). Mutation of the gene encoding BTK in humans leads to X-linked agammaglobulinemia which is characterized by reduced immune function, including impaired maturation of B cells, decreased levels of immunoglobulin and peripheral B cells, diminished T-cell independent immune response (Rosen F. S. et al., N. Engl. J. Med., 1995, 333:431-440; and Lindvall J. M. et al., Immunol. Rev. 2005, 203:200-215). Btk is activated by Src-family kinases and phosphorylates PLC gamma leading to effects on B-cell function and survival. Additionally, Btk is important in signal transduction in response to immune complex recognition by macrophage, mast cells and neutrophils. Btk inhibition is also important in survival of lymphoma cells (Herman SEM., Blood, 2011, 117:6287-6289) suggesting that inhibition of Btk may be useful in the treatment of lymphomas. As such, inhibitors of Btk and related kinases are of great interest as anti-inflammatory as well as anti-cancer agents. Btk is also important for platelet function and thrombus formation suggesting that Btk-selective inhibitors may prove to be useful antithrombotic agents (Liu J., Blood, 2006, 108:2596-603).

Bmx, another Tec family member which has roles in inflammation, cardiovascular disease, and cancer (Cenni B. et al. Int. Rev. Immunol., 2012, 31: 166-173) is also important for self-renewal and tumerogenic potential of glioblastoma stem cells (Guryanova O. A. et al. Cancer Cell 2011, 19: 498-511). As such, Bmx inhibitors are expected to be useful in the treatment of various diseases including cancer, cardiovascular disease and inflammation.

The SRC family of tyrosine kinases includes cSRC, Lyn, Fyn, Lck, Hck, Fgr, Blk, Syk, Yrk, and Yes. cSRC is critically involved in signaling pathways involved in cancer and is often over-expressed in human malignancies (Kim L. C. et al. (2009) Nat. Rev. Clin. Oncol. 6(10):587-9). cSRC is involved in signaling downstream of growth factor receptor tyrosine kinases and regulates cell cycle progression suggesting that cSRC inhibition would impact cancer cell proliferation. Furthermore, Src inhibitors or downregulation of Hck sensitize tumor cells to immunotoxins (Lui X. F., Mol. Cancer Ther. 2013, Oct. 21).

Inhibition of SRC family members may be useful in treatments designed to modulate immune function. SRC family members, including Lck, regulate T-cell receptor signal transduction which leads to gene regulation events resulting in cytokine release, survival and proliferation. Thus, inhibitors of Lck may be useful immunosuppressive agents with potential application in graft rejection and T-cell mediated autoimmune disease (Martin et al., Expert Opin. Ther. Pat. 2010, 20:1573-93). The Src family member HCK is implicated in regulation of cytokine production suggesting that inhibition of this kinase may be useful in treatment of inflammatory disease (Smolinska M. J. et al., J. Immunol. 2011; 187:6043-51). Additionally, the Src family kinase Fgr is critical for activation of mast cells and IgE-mediated anaphylaxis suggesting that this kinase is a potential therapeutic target for allergic diseases (Lee J. H. et al., J. Immunol. 2011, 187:1807-15)

Inhibition of kinases using small molecule inhibitors has successfully led to several approved therapeutic agents used in the treatment of a variety of diseases disorders and conditions. Herein, we disclose a novel family of kinase inhibitors. Further, we demonstrate that modifications in compound substitution can influence kinase selectivity and therefore the biological function of that agent.

### SUMMARY OF THE INVENTION

The present invention relates to a novel family of kinase inhibitors. Compounds of this class have been found to have inhibitory activity against members of the Tec or Scr protein kinase families.

One aspect of the present invention is directed to a compound of Formula I:

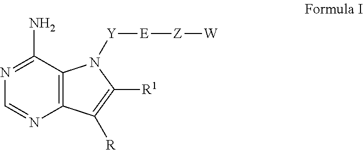

or pharmaceutically acceptable salts, solvates, solvates of salt, stereoisomers, tautomers, isotopes, prodrugs, complexes or biologically active metabolites thereof, wherein
R is selected from the group consisting of:
1) hydrogen,
2) alkyl,
3) heteroalkyl,
4) carbocyclyl,
5) heterocyclyl,
6) aryl, or
7) heteroaryl,
   wherein the alkyl, heteroalkyl, carbocyclyl, heterocyclyl, aryl, or heteroaryl are optionally substituted;

R¹ is selected from the group consisting of:
1) hydrogen,
2) alkyl,
3) heteroalkyl,
4) carbocyclyl,
5) heterocyclyl, or
6) halogen,
   wherein the alkyl, heteroalkyl, carbocyclyl, or heterocyclyl are optionally substituted;

### Y is

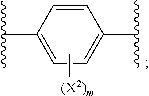

E is oxygen;

### Z is

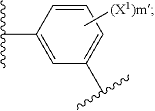

W is selected from
1) —OCH₂R², or
2) —CH₂OR²,
   wherein Y-E-Z-W is

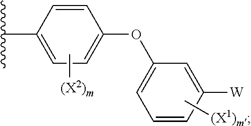

R² is selected from substituted or unsubstituted aryl, substituted or unsubstituted heteroaryl;
X¹ and X² are independently selected from hydrogen or halogen;
m is an integer from 0 to 4;
m′ is an integer from 0 to 4.

Another embodiment of the present invention includes compounds of Formula I, wherein W is selected from the group consisting of:

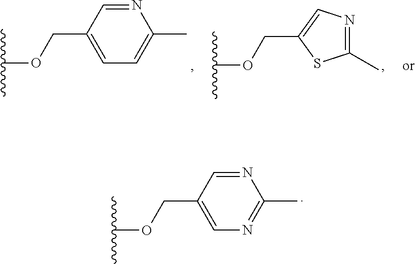

Another embodiment of the present invention includes compounds of Formula I, wherein Z is

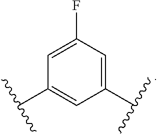 [SMILES: *c1cc(*)cc(F)c1]

Another embodiment includes compounds of Formula I, wherein Y is

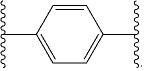 [SMILES: *c1ccc(*)cc1]

Preferred embodiment includes compounds of Formula I, wherein R¹ is hydrogen.

Another embodiment of the present invention includes compounds of Formula I, wherein R is selected from the group consisting of:

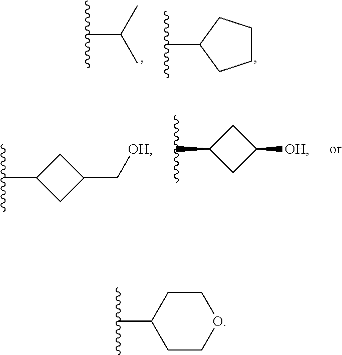

Another embodiment of the present invention includes compounds of Formula II:

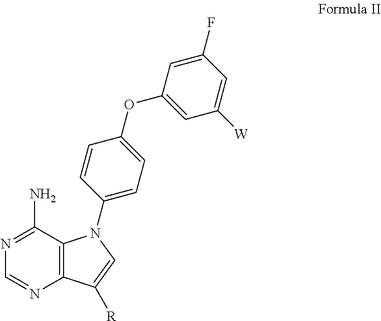

or a pharmaceutically acceptable salts, solvates, solvates of salts, stereoisomers, tautomers, isotopes, prodrugs, complexes or biologically active metabolites thereof, wherein R is selected from the group consisting of:
1) hydrogen,
2) alkyl,
3) heteroalkyl,
4) carbocyclyl,
5) heterocyclyl,
6) aryl, or
7) heteroaryl,
   wherein the alkyl, heteroalkyl, carbocyclyl, heterocyclyl, aryl, or heteroaryl are optionally substituted; and
   W is selected from the group consisting of: —OCH₂R², or —CH₂OR²,
   wherein R² is selected from substituted or unsubstituted aryl, substituted or unsubstituted heteroaryl.

Another embodiment of the present invention includes compounds of Formula II, wherein R is

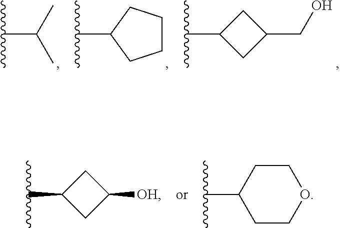

Another embodiment of the present invention includes compounds of Formula II, wherein W is

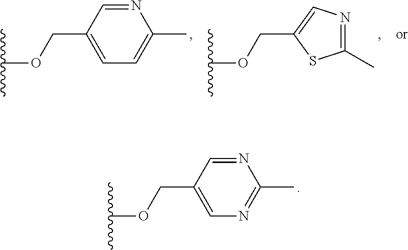

Another aspect of the present invention, provides intermediates and their synthesis related to a process of production of compounds of the invention as defined herein, or a pharmaceutically acceptable salt or solvate thereof, or a pharmaceutical composition as defined herein.

In another aspect, the present invention relates to a process for preparing a compound of Formula I, or Formula II, wherein the process comprises:

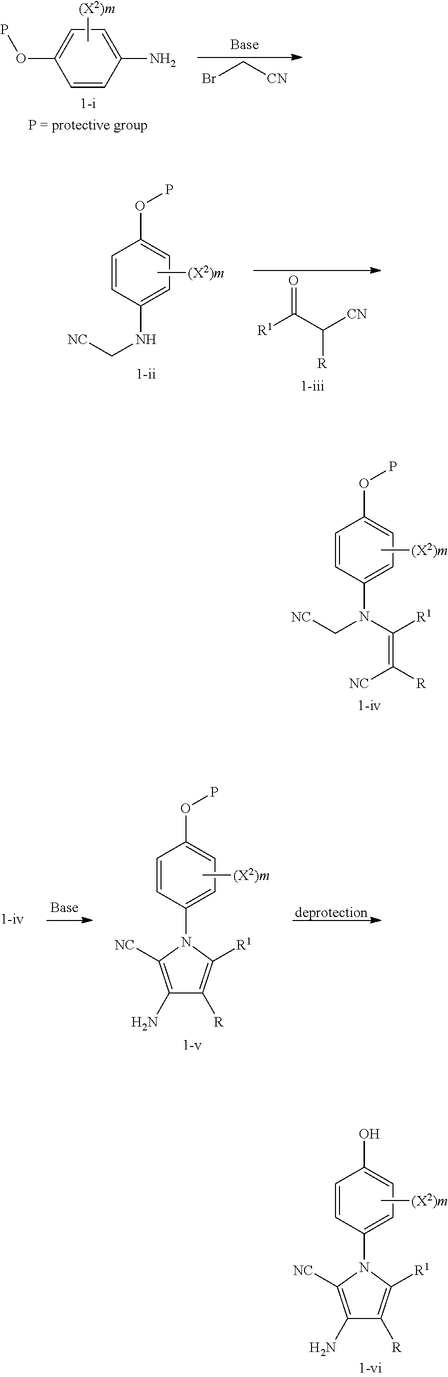

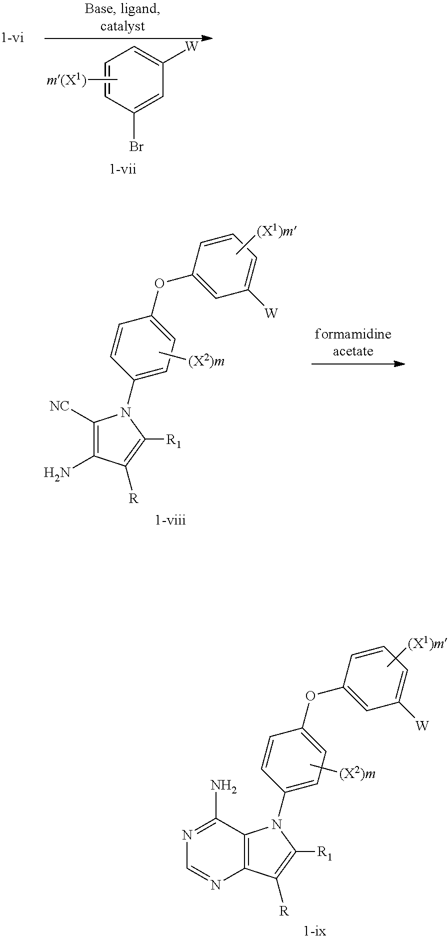

Another aspect of the present invention provides a pharmaceutical composition comprising a compound of Formula I, Formula II, or a pharmaceutically acceptable salts, solvates, solvates of salts, stereoisomers, tautomers, isotopes, prodrugs, complexes or biologically active metabolites thereof, and at least one pharmaceutically acceptable carrier, diluent, or excipient.

In another aspect, the present invention relates to a compound of the invention as defined herein, or a pharmaceutically acceptable salt, solvate, solvates of salts, stereoisomers, tautomers, isotopes, prodrugs, complexes or biologically active metabolites thereof, for use in therapy.

In another aspect, the present invention relates to a compound of the invention as defined herein, or a pharmaceutically acceptable salt or solvate thereof, or a pharmaceutical composition as defined herein, for use in the treatment of subjects suffering from a protein kinase mediated diseases or conditions.

Another aspect of the present invention provides a use of the compound of Formula I or Formula II, as an inhibitor of protein kinase, more particularly, as an inhibitor of members of the Src, or Tec family of kinases.

A further aspect of the present invention provides a use of the compound of Formula I or Formula II, as an inhibitor of protein kinase, more particularly, as an inhibitor of members of the Src, or Tec family of kinases.

In another aspect, the present invention relates to the use of a compound of the invention as defined herein, or a pharmaceutically acceptable salt, or solvate thereof, in the production of a medicament for use in the treatment of subjects suffering from a protein kinase mediated disease or condition.

A further aspect of the present invention provides a pharmaceutically acceptable salt, or solvate thereof, for use in manufacturing of a pharmaceutical composition for use in treatment of proliferative, inflammatory, infectious, or autoimmune diseases.

Another aspect of the present invention provides a compound, or pharmaceutically acceptable salts or solvates thereof, or a pharmaceutical composition as defined in present invention, for use in the treatment of a proliferative disorder, inflammatory or autoimmune disease. In a particular embodiment, the proliferative disorder, inflammatory or autoimmune disease is cancer. More particular, is a human cancer.

A further aspect of the present invention provides the use of a compound, or a pharmaceutically acceptable salt or solvate thereof, in the manufacture of a medicament for use in the treatment of a proliferative disorder, such as cancer.

Another aspect of the present invention provides a compound of Formula I, or Formula II, or a pharmaceutically acceptable salts, solvates, solvates of salts, stereoisomers, tautomers, isotopes, prodrugs, complexes or biologically active metabolites thereof, for use in the treatment of a proliferative, inflammatory, infectious, or autoimmune diseases disorder, or state in combination with an agent selected from: an estrogen receptor modulator; an androgen receptor modulator; a retinoid receptor modulator; a cytotoxic agent; an anti-proliferative agent comprises: adriamycin, dexamethasone, vincristine, cyclophosphamide, fluorouracil, topotecan, taxol, interferons, or platinum derivatives; an anti-inflammatory agent comprises: corticosteroids, TNF blockers, IL-1 RA, azathioprine, cyclophosphamide, or sulfasalazine; a prenyl-protein transferase inhibitor; an HMG-CoA reductase inhibitor; an HIV protease inhibitor; a reverse transcriptase inhibitor; an angiogenesis inhibitor comprises: sorafenib, sunitinib, pazopanib, or everolimus; an immunomodulatory or immunosuppressive agents comprises: cyclosporin, tacrolimus, rapamycin, mycophenolate mofetil, interferons, corticosteroids, cyclophophamide, azathioprine, or sulfasalazine; a PPAR-γ agonist comprising thiazolidinediones; a PPAR-δ agonist; an inhibitor of inherent multidrug resistance; an agent for the treatment of anemia, comprising: erythropoiesis-stimulating agents, vitamins, or iron supplements; an anti-emetic agent including 5-HT3 receptor antagonists, dopamine antagonists, NK1 receptor antagonist, H1 histamine receptor antagonists, cannabinoids, benzodiazepines, anticholinergic agents, or steroids; an agent for the treatment of neutropenia; an immunologic-enhancing agents; a proteasome inhibitors; an HDAC inhibitors; an inhibitor of the chemotrypsin-like activity in the proteasome; a E3 ligase inhibitors; a modulator of the immune system including interferon-alpha, Bacillus Calmette-Guerin (BCG), or ionizing radition (UVB) that can induce the release of cytokines, interleukins, TNF, or induce release of death receptor ligands including TRAIL; a modulator of death receptors TRAIL, or TRAIL agonists including humanized antibodies HGS-ETR1, or HGS-ETR2; neurotrophic factors selected from the group of: cetylcholinesterase inhibitors, MAO inhibitors, interferons, anti-convulsants, ion channel blockers, or riluzole; anti-Parkinsonian agents comprising anticholinergic agents, or dopaminergic agents, including dopaminergic precursors, monoamine oxidase B inhibitors, COMT inhibitors, dopamine receptor agonists; agents for treating cardiovascular disease comprises beta-blockers, ACE inhibitors, diuretics, nitrates, calcium channel blockers, or statins; agents for treating liver disease comprises corticosteroids, cholestyramine, or interferons; anti-viral agents, including nucleoside reverse transcriptase inhibitors, non-nucleoside reverse transcriptase inhibitors, protease inhibitors, integrase inhibitors, fusion inhibitors, chemokine receptor antagonists, polymerase inhibitors, viral proteins synthesis inhibitors, viral protein modification inhibitors, neuraminidase inhibitors, fusion or entry inhibitors; agents for treating blood disorders comprising: corticosteroids, anti-leukemic agents, or growth factors; agents for treating immunodeficiency disorders comprising gamma globulin, adalimumab, etarnecept, or infliximab; a HMG-CoA reductase inhibitors including torvastatin, fluvastatin, lovastatin, pravastatin, rosuvastatin, simvastatin, or pitavastatin, or in combination, or sequentially with radiation, or at least one chemotherapeutic agents.

More preferably the medicament is for the treatment of a proliferative disorder or disease state in combination with a death receptor agonist.

Another aspect of the present invention provides a compound, or pharmaceutically acceptable salts, or solvates thereof, or a pharmaceutical composition as defined in present invention, for use in the treatment of diseases, or disorders selected from: cancer, myeloproliferative disorders, lung fibrosis, hepatic fibrosis, cardiovascular diseases: cardiac hypertrophy, cardiomyopathy, restenosis; thrombosis, heart attacks, or stroke; alopecia, emphysema; atherosclerosis, psoriasis, or dermatological disorders, lupus, multiple sclerosis, macular degeneration, asthma, reactive synoviotides, viral disorders; CNS disorders; auto-immune disorders: glomerulonephritis, or rheumatoid arthritis; hormone-related diseases, metabolic disorders; inflammatory diseases; infectious or fungal diseases, malaria, or parasitic disorders.

Another aspect of the present invention provides a compound, or pharmaceutically acceptable salts, or solvates thereof, or a pharmaceutical composition as defined in present invention, for use in the manufacture of a medicament for use in the treatment of: arthritis, tenosynovial giant cell tumour, pigmented villonodular synovitis, or other reactive synoviotides, bone metastases formation, or progression, acute myeloid leukemia, or human cancer, or select subsets of cancer, for example breast tumours, or gastric cancer by inhibition of kinase activity.

In another aspect, the present invention relates to a method of treating a disease or condition associated with protein kinase activity, said method comprising administering to a subject a therapeutically effective amount of a compound of the invention as defined herein, or a pharmaceutically acceptable salt or solvate thereof, or a pharmaceutical composition as defined herein.

In another aspect, the present invention provides a method of treating a proliferative disorder, said method comprising administering to a subject a therapeutically effective amount of a compound, or a pharmaceutically acceptable salt, or solvate thereof, or a pharmaceutical composition as defined herein. In a particular embodiment, the proliferative disorder is a cancer.

Another aspect of the present invention provides a method of modulating kinase function, the method comprising contacting a cell with a compound of the present invention in an amount sufficient to modulate the enzymatic activity of a given kinase or kinases, from Src, or Tec family kinases, thereby modulating the kinase function.

A further aspect of the present invention provides a method of inhibiting cell proliferation, or survival in vitro or in vivo, said method comprising contacting a cell with an effective amount of a compound as defined herein, or a pharmaceutically acceptable salt, or solvate thereof.

In one embodiment, the present invention provides a method of producing a protein kinase inhibitory effect in a cell or tissue, said method comprising contacting the cell or tissue with an effective amount of a compound, or a pharmaceutically acceptable salt or solvate thereof.

In other embodiment, the present invention provides a method of producing a protein kinase inhibitory effect in vivo, said method comprising administering to a subject an effective amount of a compound, or a pharmaceutically acceptable salt, or solvate thereof. The administration may be by any suitable route of administration, such as parenteral or oral. The dosage unit may be any suitable amount, for example, the dosage unit for parenteral or oral administration may contain from 50 mg to 5000 mg of a compound of Formula I, or Formula II, or a pharmaceutical acceptable salt, or solvate thereof. The compound of the present invention may be administered 1 to 4 times a day. A dosage of between 0.01-100 mg/kg body weight/day of the compound of the present invention can be administered to a patient receiving these compositions.

The compounds of the present invention may be used alone or in combination with one or more other therapeutic agents. The combination may be achieved by way of the simultaneous, sequential, or separate dosing of the individual components of treatment. Such combination products employ the compounds of this invention, within the dose range described hereinbefore and the other pharmaceutically active agent within its approved dose range.

Another aspect of the present invention provides a method of modulating the target kinase function. The method comprising:

a) contacting a cell with a compound of the present invention in an amount sufficient to modulate the target kinase function, thereby
b) modulating the target kinase activity and signaling.

The present invention further provides a method of synthesising a compound, or a pharmaceutically acceptable salt, or solvate thereof, as defined herein.

Another aspect of the present invention provides a probe, the probe comprising a compound of Formula I, or Formula II, labeled with a detectable label, or an affinity tag. In other words, the probe comprises a residue of a compound of Formula I, or Formula II, covalently conjugated to a detectable label. Such detectable labels include, but are not limited to, a fluorescent moiety, a chemiluminescent moiety, a paramagnetic contrast agent, a metal chelate, a radioactive isotope-containing moiety, or biotin.

### DETAILED DESCRIPTION OF PREFERRED EMBODIMENTS

The present invention relates to novel kinase inhibitors. These compounds are found to have activity as inhibitors of protein kinases, including members of the Src, or Tec kinase families.

Compounds of the present invention are formulated into a pharmaceutical composition which comprises an effective amount of a compound of the present invention with at least one pharmaceutically acceptable diluent, carrier, or excipient.

The term “pharmaceutically effective amount” refers to an amount of the composition for the prevention and treatment of humans or animals that is effective in treating of disease, disorder, or condition associated with protein kinase activity.

### Pharmaceutical Compositions

According to the present invention there is provided a pharmaceutical composition which comprises a compound of Formula I, Formula II, or a pharmaceutically acceptable salt, solvate, solvate of salt, stereoisomer, tautomer, isotope, prodrug, complex or biologically active metabolite thereof, or mixtures of the compounds of the present invention, in association with at least one pharmaceutically acceptable diluent, carrier, or excipient.

The pharmaceutical compositions may be in a conventional pharmaceutical form suitable for oral administration (e.g., tablet, capsule, granules, powder, liquid solution, suspension, or syrup); parenteral administration ((including cutaneous, subcutaneous, intramuscular, intraperitoneal, intravenous, intra-arterial, intra-cerebral, intraocular injection, or infusion); suppository (rectal or vaginal); bronchial, nasal, topical, buccal, sub-lingual, transdermal, or those in a form suitable for administration by inhalation or insufflations, including powders and liquid aerosol administration, drop infusion preparations, eye lotion, or by sustained release systems. Regardless of the route of administration selected, the compounds may be formulated into pharmaceutically acceptable dosage forms by conventional methods known to those skilled in the art.

In the development of a dosage form formulation, the choice of the core excipients is extremely important. Several aspects of the finished dosage form must be considered such as the nature of the active pharmaceutical ingredient (API), the intended delivery method of the API (immediate release, modified, sustained, extended, delayed release etc), and the manufacturing process.

A non-limiting list of pharmaceutical compositions comprising a compound of Formula I, or Formula II (or combinations of the inventive compounds), according to the present invention, and at least one pharmaceutically acceptable excipient, such as a binder, a disintegrating agent, a lubricant, a diluents, a solubilizing agent, an emulsifier, a coating agent, a cyclodextrin or buffer, for use in formulation of suitable release dosage forms: “prolonged release”, “extended release”, “modified release”, “delayed release”, “sustained release” or “immediate release”, “orally disintegrating tablets”, or “sustained release parenteral depot” pharmaceutical compositions.

There are different dosage forms with plurality of “controlled release” pharmaceutical compositions, particularly “prolonged release”, “extended release”, “modified release”, “delayed release”, or “sustained release” compositions. Examples for controlled release pharmaceutical compositions are immediate release pharmaceutical compositions, enteric coated pharmaceutical compositions, pulsed release pharmaceutical compositions, or sustained release pharmaceutical compositions.

An oral controlled release pharmaceutical composition means a pharmaceutical composition including at least one active pharmaceutical ingredient which is formulated with at least one pharmaceutically acceptable film forming polymer and optionally with at least one pharmaceutically acceptable excipient, where the pharmaceutical composition shows a pH-dependent, or a pH-independent reproducible release profile.

The term “oral controlled release pharmaceutical composition”, as referred to herein, is defined to mean oral pharmaceutical compositions, which when administered releases the active ingredient at a relatively constant rate and provide plasma concentrations of the active ingredient that remain substantially invariant with time within the therapeutic range of the active ingredient over a 24-hour period and encompasses “prolonged release”, “extended release”, “modified release”, “delayed release”, or “sustained release” compositions.

The term “modified release” as referred to herein, means that the escape of the drug from the tablet has been modified in some way. Usually this is to slow the release of the drug so that the medicine doesn't have to be taken too often and therefore improves compliance. The other benefit from modifying release is that the drug release is controlled and there are smaller peaks, and troughs in blood levels therefore reducing the chance of peak effects, and increasing the likelihood of therapeutic effectiveness for longer periods of time.

The term “continuous release”, means that a term applied to a drug that is designed to deliver a dose of a medication over an extended period. The most common device for this purpose is a soft, soluble capsule containing minute pellets of the drug for release at different rates in the GI tract, depending on the thickness and nature of the oil, fat, wax, or resin coating on the pellets. Another system consists of a porous plastic carrier impregnated with the drug and a surfactant to facilitate the entry of GI fluids that slowly leach out of the drug. Ion exchange resins that bind to drugs and liquids containing suspensions of slow-release drug granules are also used to provide medication over an extended period.

The term “pulsatile release” means that a drug is delivered in one or more doses that fluctuate between a maximum and minimum dose over a predetermined time intervals. This can be represented by a dose release profile, having one or more distinct peaks or valleys. However, two or more pulsed releases may produce an overlapping, overall, or composite release profile that appears, or effectively is constant. The need for pulsatile release may include the desire to avoid drug degradation in the stomach or first pass metabolism. Pulsatile release can be achieved via coating of multiparticulates with pH dependent and/or barrier membrane coating systems, followed by blending of the multiparticulates to achieve desired release profiles.

The term “delayed” release” refers to the onset of release in relationship to administration of the drug. “Delayed” means that the release of drug is postponed, and begins, or is triggered some period of time after administration (e.g., the lag time), typically a relatively long period of time, e.g. more than one hour.

The term “immediate release” as referred to herein, is defined to mean oral pharmaceutical compositions, which when administered releases the active ingredient within a small period of time, typically less than 45 minutes after administration. Oral formulation for immediate release drug delivery system, is a conventional type of drug delivery system, which is designed to disintegrate, and release their pharmaceutically active ingredient with no rate controlling features, such as special coatings and other techniques.

The term “Orally Disintegrating Tablets” (ODT), refers to the tablet that have a disintegration time less than 60 seconds, with good mouth feel and friability that did not exceed 1%. Orally Disintegrating Tablet (ODT) allows to improve patient compliance, in particular with pediatric, geriatric, and institutionalized patients, or patients with chemotherapy-induced nausea.

Oral dosage forms which may be employed with the present invention include: tablets, granules, spheroids, or pellets in a capsule, or in any other suitable solid form.

A “depot formulation” may be formulated to provide slow absorption of the molecules of Formula I, or Formula II, or combinations thereof, or pharmaceutically acceptable salts, derivatives, isomers, polymorphs, solvates, hydrates, analogues, enantiomers, tautomeric forms, or mixtures thereof from the site of administration, often keeping therapeutic levels of the molecule or an active metabolite, in the patient's system for days or weeks at a time. Alternatively, a “depot formulation” may provide convenience for a patient in need of chronic medication by a delivering molecule of the present invention without exposure to the GI tract. Moreover, a “depot formulation” may provide better compliance due to the infrequent dosing regimen and convenience. Additional characteristics of a “depot formulation” that will enhance patient compliance are good local tolerance at the injection site and ease of administration.

Although, the dosage form will vary depending on the symptoms, age and body weight of the patient, the nature and severity of the disorder to be treated, or prevented, the route of administration, and the form of the drug. In general a daily dosage form 0.01 to 2000 mg of the compound is recommended for an adult human patient, and this may be administered in a single dose, or in divided doses. The amount of active ingredient which can be combined with a carrier material to produce a single dosage form will generally be that amount of the compound, which produces a therapeutic effect.

The time of administration and/or amount of the composition that will yield the most effective results in terms of efficacy of treatment in a given patient will depend upon the activity, pharmacokinetics, and bioavailability of a particular compound, physiological condition of the patient (including age, sex, disease type, and stage, general physical condition, responsiveness to a given dosage form, and type of medication), route of administration, etc.

The term “pharmaceutically acceptable”, is employed herein to refer to those ligands, materials, compositions, or dosage forms which are, within the scope of sound medical judgment, suitable for use in contact with the tissues of human beings and animals without excessive toxicity, irritation, allergic response, or other problem, or complication, commensurate with a reasonable benefit/risk ratio.

The term “pharmaceutically acceptable carrier”, as used herein means a pharmaceutically acceptable material, composition, or vehicle, such as a liquid or solid filler, diluent, excipient, solvent, or encapsulating material. Each carrier must be acceptable in the sense of being compatible with the other ingredients of the formulation, including the active ingredient, and not injurious or harmful to the patient. Some examples of materials which can serve as pharmaceutically acceptable carriers include: sugars, such as lactose, glucose, or sucrose; starches, such as corn starch, potato starch, and substituted or unsubstituted β-cyclodextrin; cellulose, or its derivatives, such as sodium carboxymethyl cellulose, ethyl cellulose, or cellulose acetate; powdered tragacanth; malt; gelatin; talc; or other excipients, such as cocoa butter, or suppository waxes; oils, such as peanut oil, cottonseed oil, safflower oil, sesame oil, olive oil, corn oil, or soybean oil; glycols, such as propylene glycol; polyols, such as glycerin, sorbitol, mannitol, or polyethylene glycol; esters, such as ethyl oleate, or ethyl laurate; agar; buffering agents, such as magnesium hydroxide, or aluminum hydroxide; alginic acid; pyrogen-free water; isotonic saline; Ringer's solution; ethyl alcohol; phosphate buffer solutions; and other non-toxic compatible substances employed in pharmaceutical formulations.

The term “pharmaceutically acceptable salt” refers to the relatively non-toxic, inorganic and organic acid addition salts of the compound(s). These salts can be prepared in situ during the final isolation and purification of the compound(s), or by separately reacting a purified compound(s) in its free base form, with a suitable organic or inorganic acid, and isolating the salt thus formed. Representative salts include the hydrobromide, hydrochloride, sulfate, bisulfate, phosphate, nitrate, acetate, valerate, oleate, palm itate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like (see, for example, Berge et al. (1977) “Pharmaceutical Salts”, J. Pharm. Sci. 66: 1-19).

The term “halo” or “halogen” refers to chlorine, bromine, fluorine, or iodine. Fluorine is a preferred halogen.

The pharmaceutical compositions of the present invention are obtained by conventional procedures using conventional pharmaceutical excipients, well known in the art.

In other cases, the compounds of the present invention may contain one or more acidic functional groups and, thus, are capable of forming pharmaceutically acceptable salts with pharmaceutically acceptable bases. These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, or aluminum salts, and the like. Representative organic amines useful for the formation of base addition salts include ethylamine, diethylamine, ethylenediamine, ethanolamine, diethanolamine, piperazine, and the like (see, for example, Berge et al., 1977, “Pharmaceutical Salts”).

As used herein, the term “affinity tag” means a ligand or group, linked either to a compound of the present invention, or to a protein kinase domain, that allows the conjugate to be extracted from a solution.

The term “alkyl” refers to substituted or unsubstituted saturated hydrocarbon groups, including straight-chain alkyl and branched-chain alkyl groups, including haloalkyl groups, such as trifluoromethyl and 2,2,2-trifluoroethyl, etc. Representative alkyl groups include methyl, ethyl, n-propyl, isopropyl, n-butyl, t-butyl, isobutyl, sec-butyl, (cyclohexyl)methyl, cyclopropylmethyl, n-pentyl, n-hexyl, n-heptyl, n-octyl, and the like.

The terms “alkenyl” and “alkynyl” refer to substituted or unsubstituted unsaturated aliphatic groups analogous in length, and possible substitution to the alkyls described above, but that contain at least one double or triple bond respectively. Representative alkenyl groups include vinyl, propen-2-yl, crotyl, isopenten-2-yl, 1,3-butadien-2-yl), 2,4-pentadienyl, or 1,4-pentadien-3-yl. Representative alkynyl groups include ethynyl, 1- and 3-propynyl, or 3-butynyl. In certain preferred embodiments, alkyl substituents are lower alkyl groups, e.g., having from 1 to 6 carbon atoms. Similarly, alkenyl and alkynyl preferably refer to lower alkenyl, or alkynyl groups, e.g., having from 2 to 6 carbon atoms. As used herein, “alkylene” refers to an alkyl group with two open valencies (rather than a single valency), such as —(CH₂)₁₋₁₀— and substituted variants thereof.

The term “alkoxy”, refers to an alkyl group having an oxygen attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “alkoxyalkyl” refers to an alkyl group substituted with an alkoxy group, thereby forming an ether.

The terms “amide” and “amido” are art-recognized as an amino-substituted carbonyl and include a moiety that can be represented by the general formula:

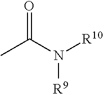

wherein R⁹, R¹⁰ are as defined above. Preferred embodiments of the amide will not include imides, which may be unstable.

The terms “amine” and “amino” are art-recognized, and refer to both unsubstituted and substituted amines, and salts thereof, e.g., a moiety that can be represented by the general formula:

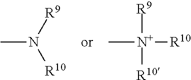

wherein R⁹, R¹⁰ and R^{10′} each independently represent a hydrogen, an alkyl, an alkenyl, —(CH₂)ₚ—R⁸, or R⁹ and R¹⁰ taken together with the N atom to which they are attached complete a heterocycle having from 4 to 8 atoms in the ring structure; R⁸ represents an aryl, a cycloalkyl, a cycloalkenyl, a heterocyclyl, or a polycyclyl; and p is zero, or an integer from 1 to 8. In preferred embodiments, only one of R⁹ or R¹⁰ can be a carbonyl, e.g., R⁹, R¹⁰, and the nitrogen together do not form an imide. In even more preferred embodiments, R⁹ or R¹⁰ (and optionally R^{10′}) each independently represent a hydrogen, an alkyl, an alkenyl, or —(CH₂)ₚ—R⁸. In certain embodiments, the amino group is basic, meaning the protonated form has a pKₐ≧7.00.

The term “aralkyl”, as used herein, refers to an alkyl group substituted with an aryl group, for example —(CH₂)ₚ—Ar.

The term “heteroaralkyl”, as used herein, refers to an alkyl group substituted with a heteroaryl group, for example —(CH₂)ₚ—Het.

The term “aryl”, as used herein includes 5-, 6-, and 7-membered substituted or unsubstituted single-ring aromatic groups in which each atom of the ring is carbon. The term “aryl” also includes polycyclic ring systems having two or more cyclic rings, in which two or more carbons are common to two adjoining rings, wherein at least one of the rings is aromatic, e.g., the other cyclic rings can be cycloalkyls, cycloalkenyls, cycloalkynyls, aryls, heteroaryls, or heterocyclyls. Aryl groups include benzene, naphthalene, phenanthrene, phenol, aniline, anthracene, or phenanthrene.

The terms “carbocycle” and “carbocyclyl”, as used herein, refer to a non-aromatic substituted or unsubstituted ring in which each atom of the ring is carbon. The terms “carbocycle” and “carbocyclyl” also include polycyclic ring systems having two or more cyclic rings, in which two or more carbons are common to two adjoining rings, wherein at least one of the rings is carbocyclic, e.g., the other cyclic rings can be cycloalkyls, cycloalkenyls, cycloalkynyls, aryls, heteroaryls, or heterocyclyls. Representative carbocyclic groups include cyclopentyl, cyclohexyl, 1-cyclohexenyl, or 3-cyclohexen-1-yl, or cycloheptyl.

The term “carbonyl”, is art-recognized and includes such moieties as can be represented by the general formula:

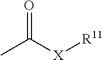

wherein X is a bond, or represents an oxygen, or a sulfur, and R¹¹ represents a hydrogen, an alkyl, an alkenyl, —(CH₂)ₚ—R⁸, or a pharmaceutically acceptable salt. Where X is oxygen, and R¹¹ is not hydrogen, the formula represents an “ester”. Where X is oxygen, and R¹¹ is hydrogen, the formula represents a “carboxylic acid”.

The term “heteroaryl”, includes substituted or unsubstituted aromatic 5- to 7-membered ring structures, more preferably 5- to 6-membered rings, whose ring structures include one to four heteroatoms. The term “heteroaryl”, also includes polycyclic ring systems having two or more cyclic rings, in which two or more carbons are common to two adjoining rings, wherein at least one of the rings is heteroaromatic, e.g., the other cyclic rings can be cycloalkyls, cycloalkenyls, cycloalkynyls, aryls, heteroaryls, or heterocyclyls. Heteroaryl groups include, for example, pyrrole, furan, thiophene, imidazole, isoxazole, oxazole, thiazole, triazole, pyrazole, pyridine, pyrazine, pyridazine or pyrimidine, and the like.

The term “heteroatom”, as used herein, means an atom of any element other than carbon or hydrogen. Preferred heteroatoms are nitrogen, oxygen, or sulfur.

The terms “heterocyclyl” or “heterocyclic group”, refer to substituted or unsubstituted non-aromatic 3- to 10-membered ring structures, more preferably 3- to 7-membered rings, whose ring structures include one to four heteroatoms. The terms “heterocyclyl” or “heterocyclic group”, also include polycyclic ring systems having two or more cyclic rings, in which two or more carbons are common to two adjoining rings, wherein at least one of the rings is heterocyclic, e.g., the other cyclic rings can be cycloalkyls, cycloalkenyls, cycloalkynyls, aryls, heteroaryls, or heterocyclyls. Heterocyclyl groups include, for example, tetrahydrofuran, tetrahydropyran, piperidine, piperazine, pyrrolidine, morpholine, lactones, or lactams.

The term “hydrocarbon”, as used herein, refers to a group that is bonded through a carbon atom that does not have a ═O, or ═S substituent, and typically has at least one carbon-hydrogen bond, and a primarily carbon backbone, but may optionally include heteroatoms. Thus, groups like methyl, ethoxyethyl, 2-pyridyl, or trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, or combinations thereof.

The terms “polycyclyl” or “polycyclic”, refer to two or more rings (e.g., cycloalkyls, cycloalkenyls, cycloalkynyls, aryls, heteroaryls, and/or heterocyclyls), in which two or more carbons are common to two adjoining rings, e.g., the rings are “fused rings”. Each of the rings of the polycycle can be substituted or unsubstituted.

As used herein, the term “probe”, means a compound of the invention, which is labeled with either a detectable label, or an affinity tag, and which is capable of binding, either covalently, or non-covalently to a protein kinase domain. When, for example, the probe is non-covalently bound, it may be displaced by a test compound. When, for example, the probe is bound covalently, it may be used to form cross-linked adducts, which may be quantified and inhibited by a test compound.

The term “substituted”, refers to moieties having substituents replacing a hydrogen on one, or more atoms of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted”, is contemplated to include all permissible substituents of organic compounds. In a broad aspect, the permissible substituents include acyclic and cyclic, branched and unbranched, carbocyclic or heterocyclic, aromatic or non-aromatic substituents of organic compounds. The permissible substituents can be one or more and the same, or different for appropriate organic compounds. For purposes of this invention, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Compounds of the present invention also include isotopes of atoms present in the intermediates and/or final compounds. Isotopes include those atoms having the same atomic number, but different mass numbers. For example, isotopes of hydrogen include deuterium, or tritium.

### Therapeutic Uses and Applications

The compounds of the present invention are inhibitors of protein kinase activity.

An aspect of the present invention provides a compound of Formula I, or Formula II, or combinations thereof, or a pharmaceutically acceptable salt, or solvate, solvate of salt, stereoisomer, tautomer, isotope, prodrug, complex or biologically active metabolite thereof, for use in therapy.

The compounds of the present invention are suitable for producing a protein kinase inhibitory effect in vivo, and thus, are suitable for the treatment of diseases or conditions in which one or more of the protein kinase targets are implicated.

In one embodiment, the protein kinase is selected from the following group: Tec, Src, Abl, Jak, Csk, Fak, Syk, Fer, or Ack kinases, and receptor protein kinases. Preferably the protein kinases are from Tec, or Src kinase family.

In one embodiment, the compounds are suitable for inhibition of a proliferative disorder mediated by Tec kinase targets.

In other embodiment, the compounds are suitable for inhibition of a proliferative disorder mediated by Src kinase targets.

An aspect of the present invention provides a method of inhibiting protein kinase activity in a cell, the method comprising administering to said cell compound of Formula I or Formula II, or combinations thereof, or a pharmaceutically acceptable salt, solvate, solvate of salt, stereoisomer, tautomer, isotope, prodrug, complex or biologically active metabolite thereof.

In a further aspect, the present invention provides a method of inhibiting protein kinase in vitro or in vivo, said method comprising contacting a cell with an effective amount of a compound, or a pharmaceutically acceptable salt or solvate thereof, as defined herein.

A further aspect of the present invention provides a method of inhibiting protein kinase activity in a human or animal subject, the method comprising administering to said subject an effective amount of a compound of Formula I or Formula II, or combinations thereof, as defined herein or a pharmaceutically acceptable salt, or solvate thereof.

The compounds of the present invention are suitable for the treatment of diseases or conditions in which one or more of the protein kinase targets outlined above are implicated.

The term “proliferative disorder” is used herein in a broad sense to include any disorder that requires control of deleterious cell proliferation, for example cancers or other disorders associated with uncontrolled cellular proliferation such as dermatological disorders such as psoriasis, certain viral disorders; certain cardiovascular diseases such as restenosis, or cardiomyopathy; certain CNS disorders; auto-immune disorders such as glomerulonephritis, or rheumatoid arthritis; hormone-related diseases; metabolic disorders; thrombosis stroke, alopecia, emphysema, inflammatory diseases, or infectious diseases such fungal diseases, or parasitic disorders such as malaria. In these disorders, the compounds of the present invention may induce apoptosis, or maintain stasis within the desired cells as required.

The term “protein kinase mediated disease”, is used herein associated with abnormal cellular responses triggered by protein kinase-mediated events. Furthermore, aberrant activation or excessive expression of various protein kinases is implicated in the mechanism of multiple diseases and disorders characterized by benign, or malignant proliferation. These diseases include, but are not limited to allergies, or asthma, Alzheimer's disease, autoimmune diseases, bone diseases, cancer, cardiovascular diseases, inflammatory diseases, hormone-related diseases, metabolic diseases, neurological, or neurodegenerative diseases. Thus, inhibitors of kinase families are expected to be suitable in the treatment of cancer, vascular disease, autoimmune diseases, or inflammatory conditions including, but not limited to: solid tumors, hematological malignancies, thrombus, arthritis, graft versus host disease, lupus erythematosus, psoriasis, colitis, illeitis, multiple sclerosis, uveitis, coronary artery vasculopathy, systemic sclerosis, atherosclerosis, asthma, transplant rejection, allergy, or dermatomyositis.

In one embodiment, the compound of Formula I, Formula II, combinations thereof, or pharmaceutically acceptable salts, solvates, solvates of salts, stereoisomers, tautomers, isotopes, prodrugs, complexes, or biologically active metabolites thereof, is acting by inhibiting one or more of the host cell kinases involved in cell proliferation, cell survival, viral replication, cardiovascular disorders, neurodegeneration, autoimmunity, a metabolic disorder, stroke, alopecia, an inflammatory disease, or an infectious disease.

In another embodiment, the proliferative disorder is cancer. The cancer may be selected from the group consisting of: chronic lymphocytic leukaemia (CLL), lymphoma, leukaemia, breast cancer, lung cancer, prostate cancer, colon cancer, melanoma, pancreatic cancer, ovarian cancer, squamous carcinoma, carcinoma of head, or neck, endometrial cancer, or oesophageal carcinoma.

In another embodiment of the present invention, the infectious disease includes diseases that are caused by protozoal infestations in humans and animals. Such veterinary and human pathogenic Protozoas are preferably intracellular active parasites of the phylum Apicomplexa or Sarcomastigophora, especially Trypanosoma, Plasmodia, Leishmania, Babesia, or Theileria, Cryptosporidia, Sacrocystida, Amoebia, Coccidia, or Trichomonadia. The compounds of the present invention are particularly suitable for the treatment of Malaria tropica, caused by Plasmodium falciparum, Malaria tertiana, caused by Plasmodium vivax, or Plasmodium ovale, or for the treatment of Malaria quartana, caused by Plasmodium malariae. These compounds are also suitable for the treatment of Toxoplasmosis, caused by Toxoplasma gondii Coccidiosis caused for instance by Isospora belli, intestinal Sarcosporidiosis, caused by Sarcocystis suihominis, dysentery caused by Entamoeba histolytica, Cryptosporidiosis caused by Cryptosporidium parvum, Chagas disease, caused by Trypanosoma cruzi, sleeping sickness, caused by Trypanosoma brucei rhodesiense or gambiense, the cutaneous or visceral as well as other forms of Leishmaniosis. The present invention is also suitable for the treatment of animals infected by veterinary pathogenic Protozoa, like Theileria parva, the pathogen causing bovine East coast fever, Trypanosoma congolense, or Trypanosoma vivax, Trypanosoma brucei pathogens causing Nagana cattle disease in Africa, Trypanosoma brucei evansi causing Surra, Babesia bigemina, the pathogen causing Texas fever in cattle and buffalos, Babesia bovis the pathogen causing European bovine Babesiosis as well as Babesiosis in dogs, cats and sheep; Sarcocystis ovicanis and Sarcocystis ovifelis pathogens causing Sarcocystiosis in sheep, cattle and pigs; Cryptosporidia pathogens causing Cryptosporidioses in cattle and birds; Eimeria and Isospora species, pathogens causing Coccidiosis in rabbits, cattle, sheep, goats, pigs and birds, especially in chickens and turkeys. The compounds of the present invention is particularly preferred for use in the treatment of Coccidiosis or Malaria infections, or for the preparation of a drug, or feed stuff for the treatment of these diseases. These treatments can be prophylactic or curative. In the treatment of malaria, the protein kinase inhibitor as defined above, may be combined with at least one other anti-malaria agent. The present compound described may further be used for viral infections, or other infections caused by Pneumocystis carinii.

Tec kinases is a family of non-receptor tyrosine kinases predominantly, but not exclusively, expressed in cells of hematopoietic origin. The Tec family comprises: Tec, Bruton's tyrosine kinase (Btk), inducible T-cell kinase (Itk), resting lymphocyte kinase (Rlk/Txk), and bone marrow-expressed kinase (Bmx/Etk).

Btk is activated by Src-family kinases and phosphorylates PLC gamma leading to effects on B-cell function and survival. Additionally, Btk is important in signal transduction in response to immune complex recognition by macrophage, mast cells and neutrophils. Btk inhibition is also important in survival of lymphoma cells (Herman SEM. Blood, 2011, 117:6287-6289) suggesting that inhibition of Btk may be useful in the treatment of lymphomas. Bmx, another Tec family member are expected to be suitable in the treatment of various diseases including cancer, cardiovascular disease or inflammation.

In further aspect of the present invention, the compound of Formula I, Formula II, combinations thereof, or pharmaceutically acceptable salts, solvates, solvates of salts, stereoisomers, tautomers, isotopes, prodrugs, complexes or biologically active metabolites thereof, is acting as inhibitor of cell kinases, as anti-inflammatory, anti-cancer, or as antithrombotic agents. These compounds may be used alone, or in combination with one of more agents for the treatment of cancer, inflammatory diseases, or thrombi.

More specifically, the compounds of the present invention can also be used in combination with one or more chemotherapeutic agents, used particularly in treatment of cancer, or other neoplasms.

The compounds of Formula I, Formula II, combinations thereof, or pharmaceutically acceptable salts, solvates, solvates of salts, stereoisomers, tautomers, isotopes, prodrugs, complexes or biologically active metabolites thereof, object of the present invention can be used in combination with:
1. Anti-proliferative agents, such as adriamycin, dexamethasone, vincristine, cyclophosphamide, fluorouracil, topotecan, taxol, interferons, or platinum derivatives; anti-inflammatory agents, such as corticosteroids, TNF blockers, IL-1 RA, azathioprine, cyclophosphamide, or sulfasalazine;
2. Prenyl-protein transferase inhibitors;
3. Angiogensis inhibitors comprising sorafenib, sunitinib, pazopanib, or everolimus;
4. Immunomodulatory or immunosuppressive agents comprising: cyclosporin, tacrolimus, rapamycin, mycophenolate mofetil, interferons, corticosteroids, cyclophophamide, azathioprine, or sulfasalazine;
5. PPAR-γ agonists such as thiazolidinediones;
6. PPAR-δ agonists;
7. Inhibitors of inherent multidrug resistance;
8. Agents for the treatment of anemia, comprising erythropoiesis, stimulating agents, vitamins, or iron supplements;
9. Anti-emetic agents including 5-HT3 receptor antagonists, dopamine antagonists, NK1 receptor antagonists, H1 histamine receptor antagonists, cannabinoids, benzodiazepines, anticholinergic agents, or steroids;
10. Agents for the treatment of neutropenia;
11. Immunologic-enhancing agents;
12. Proteasome inhibitors;
13. HDAC inhibitors;
14. Inhibitors of the chemotrypsin-like activity in the proteasome;
15. E3 ligase inhibitors;
16. Modulators of the immune system including interferon-alpha, Bacillus Calmette-Guerin (BCG), or ionizing radiation (UVB) that can induce the release of cytokines, such as the interleukins, TNF, or induce release of death receptor ligands, such as TRAIL;
17. Modulators of death receptors TRAIL, or TRAIL agonists such as the humanized antibodies HGS-ETR1, or HGS-ETR, in combination, or sequentially with radiation therapy;
18. Neurotrophic factors comprising acetylcholinesterase inhibitors, MAO inhibitors, interferons, anti-convulsants, ion channel blockers, or riluzole;
19. Anti-Parkinsonian agents comprising anticholinergic agents, dopaminergic agents, including dopaminergic precursors, monoamine oxidase B inhibitors, COMT inhibitors, or dopamine receptor agonists;
20. Agents for treating cardiovascular disease, such as beta-blockers, ACE inhibitors, diuretics, nitrates, calcium channel blockers, or statins;
21. Agents for treating liver disease comprising: corticosteroids, cholestyramine, or interferons;
22. Anti-viral agents including nucleoside reverse transcriptase inhibitors, non nucleoside reverse transcriptase inhibitors, protease inhibitors, integrase inhibitors, fusion inhibitors, chemokine receptor antagonists, polymerase inhibitors, viral proteins synthesis inhibitors, viral protein modification inhibitors, neuraminidase inhibitors, fusion or entry Inhibitors;
23. Agents for treating blood disorders, such as corticosteroids, anti-leukemic agents, or growth factors;
24. Agents for treating immunodeficiency disorders, such as gamma globulin, adalimumab, etarnecept, or infliximab; or
25. HMG-CoA reductase inhibitors comprising torvastatin, fluvastatin, lovastatin, pravastatin, rosuvastatin, simvastatin, or pitavastatin.

As defined herein, an effect against a proliferative disorder mediated by a kinase within the scope of the present invention may be demonstrated by the ability to inhibit a purified kinase in vitro, or to inhibit cell proliferation, or survival in an in vitro cell assay, for example in Btk Kinase Inhibition Assay and Splenic Cell Proliferation Assay. These assays are described in more details in the accompany examples.

The present invention includes the transdermal, rectal, parenteral, or oral administration of compounds of Formula I, or Formula II, or combinations thereof, or a pharmaceutical acceptable salt, solvate, solvate of salt, stereoisomer, tautomer, isotope, prodrug, complex or biologically active metabolite thereof, to a human or animal subject. The dosage unit for the administration may contain any suitable amount of a compound of Formula I, Formula II, combinations thereof (or a pharmaceutical acceptable salt or solvate thereof, or combinations thereof), for example, from 10 mg to 5000 mg. Preferably the dosage unit for the oral administration may contain from 50 mg to 500 mg per human subject.

The compound of Formula I, or Formula II, combinations thereof, or a pharmaceutical acceptable salt or solvate thereof, of the present invention may be administered 1 to 4 times a day. A dosage may be any suitable therapeutically effective amount, for example, between 0.01-100 mg/kg body weight/day of the compound of the present invention can be administered to a patient receiving these compositions. The dose can vary within wide limits and is to be suited to the individual conditions in each individual case. For the above uses the appropriate dosage will vary depending on the mode of administration, the particular condition to be treated and the effect desired. Preferably a dose of 1 to 50 mg/kg body weight/day may be used.

In an embodiment of the present invention suitable dosage rates for larger mammals, for example humans, are of the order of from about 10 mg to 3 g/day, administered orally once, or divided doses such as 2 to 4 times a day, or in sustained release form. For topical delivery, depending on the permeability of the skin, the type and the severity of the disease, on the type of formulation, and frequency of application, different concentrations of active compounds within the medicament can be sufficient to elicit a therapeutic effect by topical application. Preferably, the concentration of an active compound pharmaceutically acceptable salts, solvates, solvates of salts, stereoisomers, tautomers, isotopes, prodrugs, complexes or biologically active metabolites thereof, within a medicament according to the present invention is in the range of between 1 μmol/L and 100 mmol/L.

### SPECIFIC ABBREVIATIONS

MS mass spectrometry
ml milliliter
μl microliter
mmol millimole
THF tetrahydrofuran
H₂ hydrogen
Pd/C palladium on carbon
PTSA p-toluenesulfonic acid
HCl hydrogen chloride
NaH sodium hydride (60% in mineral oil)
tBuOK potassium tert-butoxide
LDA lithium diisopropylamide
CuI copper (I) iodide
Cs₂CO₃ cesium carbonate
DIPEA N,N-diisopropylethylamine
MgSO₄ magnesium sulfate
NaHCO₃ sodium bicarbonate
TBAF tetra-n-butylammonium fluoride
H₂O₂ hydrogen peroxide
BH₃.Me₂S borane dimethyl sulfide complex

### Synthetic Methods

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

In further embodiment of the present invention is provided general synthetic method(s) useful in the process for preparing compounds described herein.

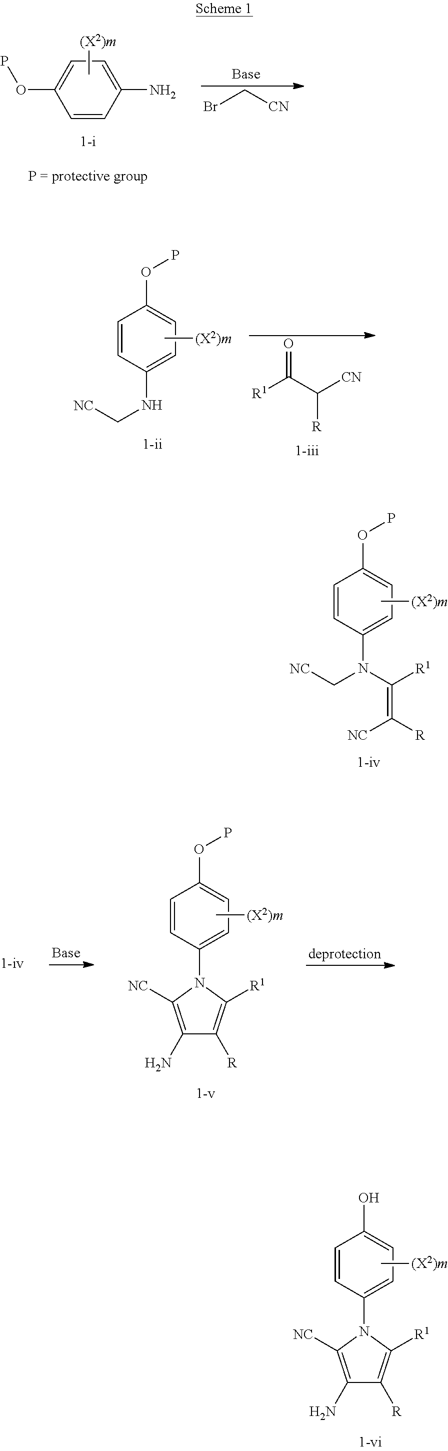

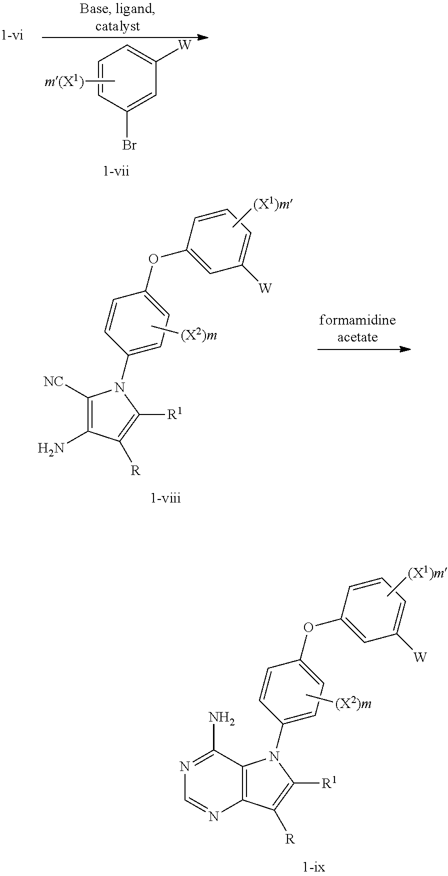

### EXAMPLES

The following synthetic methods are intended to be representative of the chemistry used to prepare compounds of the present invention and are not intended to be limiting.

### Synthesis of Intermediate 2-c

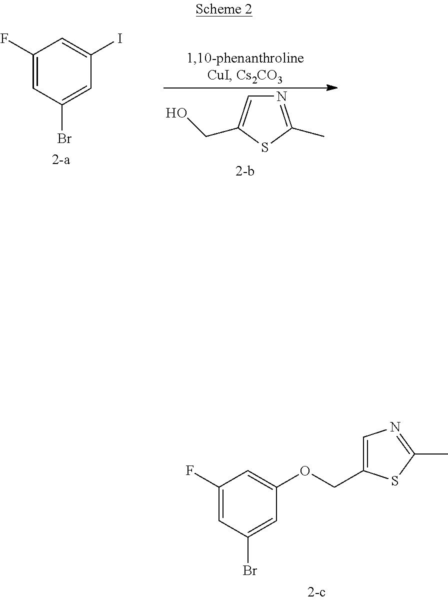

To a solution of 1-bromo-3-fluoro-5-iodobenzene 2-a (7.5 g, 25.0 mmol) in 1,4-dioxane (12.5 ml) was added (2-methylthiazol-5-yl)methanol 2-b (3.5 g, 27.5 mmol), 1,10-phenanthroline (901 mg, 5.0 mmol), copper (I) iodide (476 mg, 2.5 mmol), and cesium carbonate (11.40 g, 35.0 mmol). The reaction was stirred at 110° C. for 2 days, and then cooled to room temperature, diluted with ethyl acetate, and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered and concentrated under reduced pressure. Purification by silica gel chromatography provided Intermediate 2-c as a beige oil.

### Synthesis of Intermediate 3-b

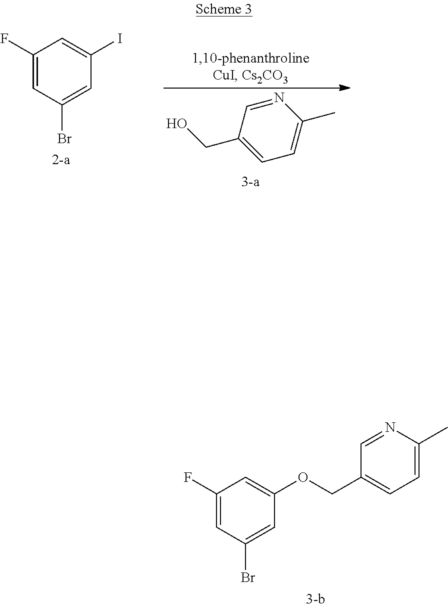

To a solution of 1-bromo-3-fluoro-5-iodobenzene 2-a (5.0 g, 16.6 mmol) in toluene (8.3 ml) was added (6-methylpyridin-3-yl) methanol 3-a (2.2 g, 18.2 mmol), 1,10-phenanthroline (599 mg, 3.3 mmol), copper (I) iodide (316 mg, 1.66 mmol), and cesium carbonate (7.6 g, 23.2 mmol). The reaction was stirred at 110° C. for 2 days, and then cooled to room temperature, diluted with ethyl acetate, and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered and concentrated under reduced pressure. Purification by silica gel chromatography provided Intermediate 3-b as a beige solid.

### Synthesis of Intermediate 4-b

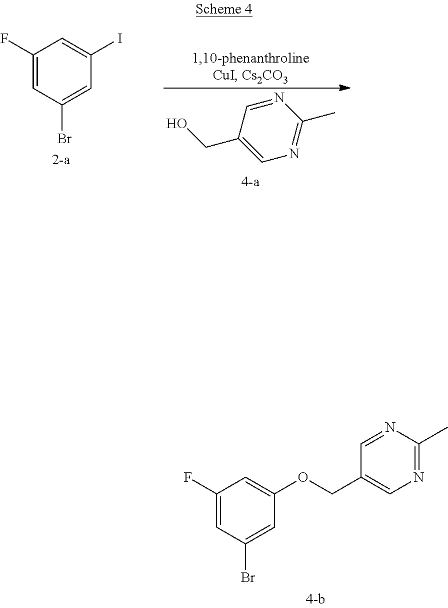

To a solution of 1-bromo-3-fluoro-5-iodobenzene 2-a (5.0 g, 16.6 mmol) in toluene (8.3 ml) was added (2-methylpyrimidin-5-yl)methanol 4-a (2.2 g, 18.3 mmol), 1,10-phenanthroline (599 mg, 3.3 mmol), copper (I) iodide (316 mg, 1.7 mmol), and cesium carbonate (7.6 g, 23.3 mmol). The reaction was stirred at 110° C. for 2 days, and then cooled to room temperature, diluted with ethyl acetate, and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered and concentrated under reduced pressure. Purification by silica gel chromatography provided Intermediate 4-b as a beige solid.

### Synthesis of Intermediate 5-b

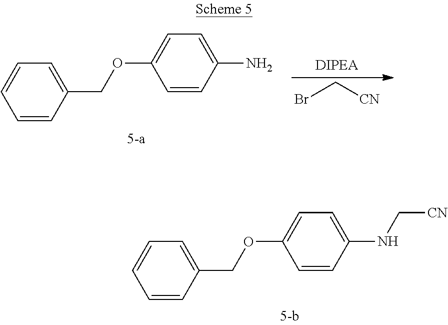

To a solution of 4-(benzyloxy)aniline, HCl 5-a (40.0 g, 170.0 mmol) and 2-bromoacetonitrile (26.7 g, 223.0 mmol) in THF (242 ml) was added DIPEA (65.2 ml, 373.0 mmol). The reaction was stirred at 80° C. overnight, and then cooled to room temperature. A saturated aqueous solution of ammonium chloride and ethyl acetate were added, the organic layer was separated and the aqueous phase was extracted twice with ethyl acetate. The combined organic extracts were washed with brine, dried over MgSO₄, filtered, and concentrated under reduced pressure. Hexanes was added to the residue, a precipitate formed and was collected by filtration to provide Intermediate 5-b as a beige solid.

### Synthesis of Intermediate 6-b

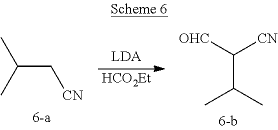

To a solution of 3-methylbutanenitrile 6-a (10.0 g, 120.0 mmol) in THF (40.2 ml) cooled to −78° C. was added drop wise a 2.0 M solution of LDA in THF (60.1 ml, 120.0 mmol). The solution was stirred for 10 minutes and then added to a solution of ethyl formate (9.4 g, 126.0 mmol) in THF (50.2 ml) cooled to −78° C. The reaction was stirred at −78° C. for 30 minutes, then slowly warmed to room temperature, and stirred overnight. The reaction was quenched by addition of 1N aqueous HCl until pH=3, and then extracted with ethyl acetate. The combined organic extracts were dried over MgSO₄, filtered, and concentrated in vacuo to provide Intermediate 6-b as a yellow oil.

### Synthesis of Intermediate 7-c

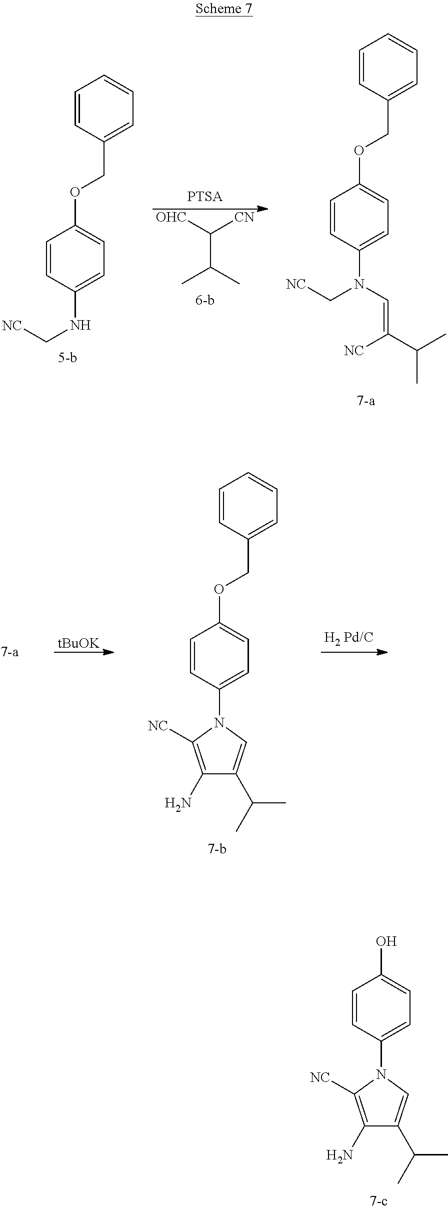

### Step 1: Intermediate 7-a

To a solution of intermediate 5-b (8.9 g, 37.5 mmol) in toluene (20 ml) was added intermediate 6-b (5.0 g, 45.0 mmol), and PTSA (713 mg, 3.7 mmol). The reaction was stirred at reflux using a Dean-Stark apparatus overnight, and then cooled to room temperature. A saturated aqueous solution of NaHCO₃ and ethyl acetate were added, the organic layer was separated, and the aqueous phase was extracted twice with ethyl acetate. The combined organic extracts were washed with brine, dried over MgSO₄, filtered, and concentrated under reduced pressure to provide Intermediate 7-a as a beige solid.

### Step 2: Intermediate 7-b

To a solution of intermediate 7-a (5.0 g, 15.1 mmol) in tert-butanol (97.0 ml) was added a 1.0 M solution of potassium tert-butoxide in tert-butanol (16.6 ml, 16.6 mmol). The reaction was stirred for 30 minutes at 80° C., then cooled to room temperature and poured in 10% aqueous HCl. Ethyl acetate was added, the organic layer was separated, washed with brine, dried over MgSO₄, filtered, and concentrated under reduced pressure. Purification by silica gel chromatography provided Intermediate 7-b as a beige solid.

### Step 5: Intermediate 7-c

To a solution of intermediate 7-b (2.8 g, 8.4 mmol) in ethyl acetate and stirred under nitrogen was added 10% Pd/C (1.8 g, 0.8 mmol). The reaction mixture was purged with H₂ and stirred for 1 hour under 1 atmosphere of hydrogen. The reaction was then filtered through celite, and the filtrate was concentrated in vacuo to provide Intermediate 7-c as a beige solid.

### Synthesis of Intermediate 8-d

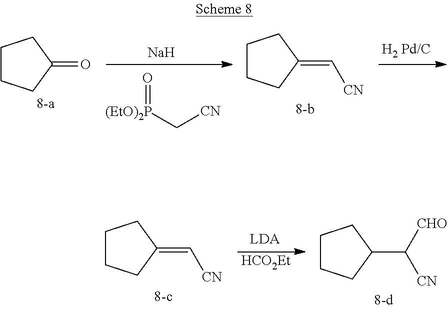

### Step 1: Intermediate 8-b

To a suspension of NaH (2.6 g, 65.4 mmol) in diethyl ether (100 ml) cooled to 0° C. was added diethyl cyanomethylphosphonate (11.58 g, 65.4 mmol) drop wise followed by a solution of cyclopentanone 8-a (5.0 g, 59.4 mmol) in diethyl ether (100 ml). After the addition was completed, the reaction was warmed to room temperature, and stirred overnight. Water and ethyl acetate were added, the organic layer was separated, washed with brine, dried over MgSO₄, filtered, and concentrated under reduced pressure to provide Intermediate 8-b as a colorless oil.

### Step 2: Intermediate 8-c

To a solution of intermediate 8-b (7.0 g, 65.3 mmol) in ethyl acetate and acetic acid (1 ml) stirred under nitrogen, was added 10% Pd/C (2.8 g, 1.32 mmol). The reaction mixture was purged with H₂, and stirred for 3 hours under 1 atmosphere of hydrogen. The reaction was then filtered through celite, and the filtrate was concentrated in vacuo to provide intermediate 8-c as a beige oil.

### Step 3: Intermediate 8-d

To a solution of intermediate 8-c (7.0 g, 64.1 mmol) in THF (21.4 ml) cooled to −78° C. was added drop wise a 2.0 M solution of LDA in THF (32.1 ml, 64.2 mmol). The solution was stirred for 10 minutes, and then added to a solution of ethyl formate (9.36 g, 126.0 mmol) in THF (50.2 ml) cooled to −78° C. The reaction was stirred at −78° C. for 30 minutes, then slowly warmed to room temperature, and stirred overnight. The reaction was quenched by addition of 1N HCl until pH=3, and then extracted with ethyl acetate. The combined organic extracts were dried over MgSO₄, filtered, and concentrated in vacuo to provide Intermediate 8-d as a yellow oil.

### Synthesis of Intermediate 9-c

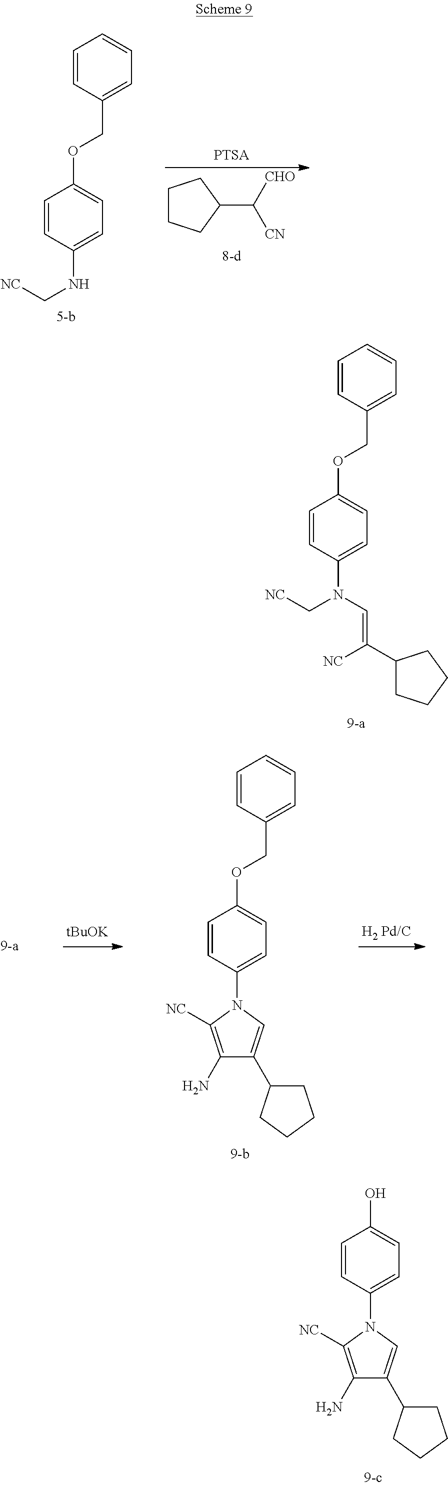 [SMILES: N#CCNc1ccc(OCc2ccccc2)cc1 ; N#CC(C=O)C1CCCC1 ; "tBuOK" ; N#CCN(/C=C(\C#N)C1CCCC1)c1ccc(OCc2ccccc2)cc1 ; N#Cc1c(N)c(C2CCCC2)cn1-c1ccc(OCc2ccccc2)cc1 ; N#Cc1c(N)c(C2CCCC2)cn1-c1ccc(O)cc1]

### Step 2: Intermediate 9-a

To a solution of Intermediate 5-b (7.2 g, 30.4 mmol) in toluene (20 ml), was added intermediate 8-d (5.0 g, 36.4 mmol), and PTSA (578 mg, 3.0 mmol). The reaction was stirred at reflux using a Dean-Stark apparatus overnight, and then cooled to room temperature. A saturated aqueous solution of NaHCO₃ and ethyl acetate were added, the organic layer was separated, the aqueous phase was extracted twice with ethyl acetate, the combined organic extracts were washed with brine, dried over MgSO₄, filtered, and concentrated under reduced pressure to provide Intermediate 9-a as a beige solid.

### Step 3: Intermediate 9-b

To a solution of Intermediate 9-a (5.0 g, 13.9 mmol) in tert-butanol (69.9 ml) was added a 1.0 M solution of potassium tert-butoxide in tert-butanol (15.4 ml, 15.4 mmol). The reaction was stirred for 30 minutes at 80° C., then cooled to room temperature, and poured in 10% aqueous HCl. Ethyl acetate was added, the organic layer was separated, washed with brine, dried over MgSO₄, filtered, and concentrated under reduced pressure. Purification by silica gel chromatography provided Intermediate 9-b as a beige solid.

### Step 4: Intermediate 9-c

To a solution of Intermediate 9-b (5.0 g, 14.0 mmol) in ethyl acetate and stirred under nitrogen was added 10% Pd/C (2.9 g, 1.4 mmol). The reaction mixture was purged with H₂ and stirred for 3 hours under 1 atmosphere of hydrogen. The reaction was then filtered through celite and the filtrate was concentrated in vacuo, to provide Intermediate 9-c as a beige solid.

### Synthesis of Intermediate 10-d

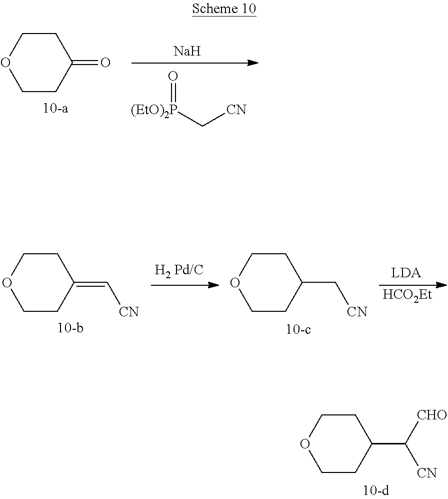

### Step 1: Intermediate 10-b

To a suspension of NaH (2.2 g, 54.9 mmol) in diethyl ether (100 ml) cooled to 0° C. was added diethyl cyanomethylphosphonate (9.7 g, 54.9 mmol) drop wise, followed by a solution of dihydro-2H-pyran-4(3H)-one 10-a (5.0 g, 59.4 mmol) in diethyl ether (100 ml). After the addition was completed the reaction was warmed to room temperature, and stirred overnight. Water and ethyl acetate were added, the organic layer was separated, washed with brine, dried over MgSO₄, filtered, and concentrated under reduced pressure, to provide Intermediate 10-b as a colorless oil.

### Step 2: Intermediate 10-c

To a solution of intermediate 10-b (6.0 g, 48.7 mmol) in ethyl acetate and acetic acid (1 ml) stirred under nitrogen was added 10% Pd/C (2.0 g, 0.9 mmol). The reaction mixture was purged with H₂ and stirred overnight under 1 atm of hydrogen. The reaction was then filtered through celite, and the filtrate was concentrated in vacuo to provide Intermediate 10-c as a beige oil.

### Step 3: Intermediate 10-d

To a solution of Intermediate 10-c (6.0 g, 47.9 mmol) in THF (16.0 ml) cooled to −78° C. was added drop wise a 2.0 M solution of LDA in THF (23.9 ml, 47.8 mmol). The solution was stirred for 10 minutes, and then added to a solution of ethyl formate (3.7 g, 50.3 mmol) in THF (20.0 ml) cooled to −78° C. The reaction was stirred at −78° C. for 30 minutes, then slowly warmed to room temperature, and stirred overnight. The reaction was quenched by addition of 1N HCl until pH=3, and then extracted with ethyl acetate. The combined organic extracts were dried over MgSO₄, filtered, and concentrated under reduced pressure to provide Intermediate 10-d as a yellow oil.

### Synthesis of Intermediate 11-c

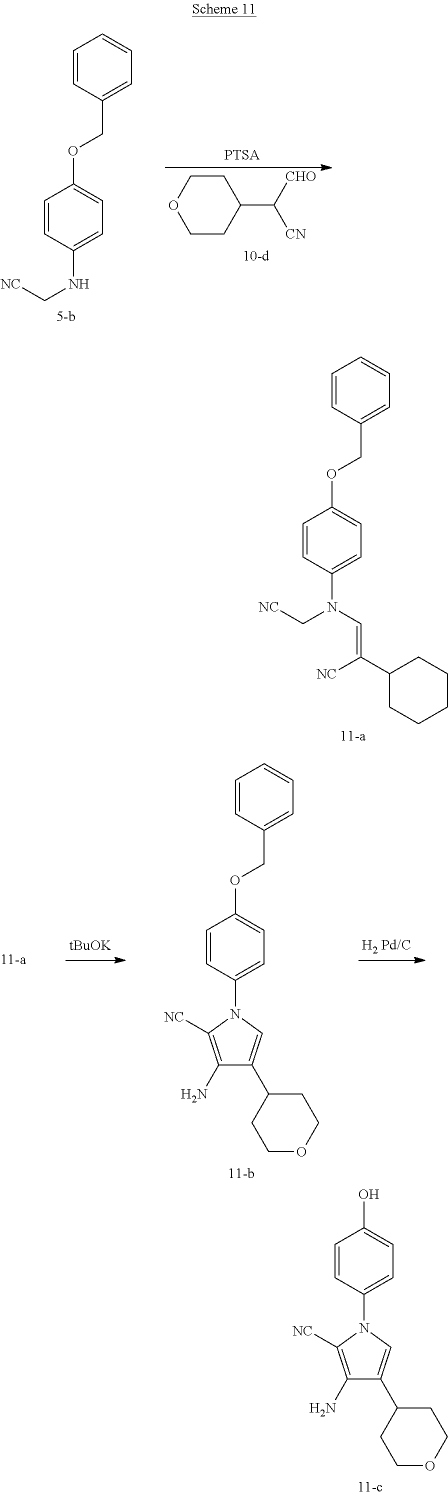 [SMILES: N#CCNc1ccc(OCc2ccccc2)cc1 ; N#CC(C=O)C1CCOCC1 ; "tBuOK" ; N#CCN(/C=C(\C#N)C1CCCCC1)c1ccc(OCc2ccccc2)cc1 ; N#Cc1c(N)c(C2CCOCC2)cn1-c1ccc(OCc2ccccc2)cc1 ; N#Cc1c(N)c(C2CCOCC2)cn1-c1ccc(O)cc1]

### Step 2: Intermediate 11-a

To a solution of Intermediate 5-b (6.9 g, 29.0 mmol) in toluene (20 ml), was added Intermediate 10-d (5.3 g, 34.7 mmol) and PTSA (551 mg, 2.9 mmol). The reaction was stirred at reflux overnight using a Dean-Stark apparatus, and then cooled to room temperature. A saturated aqueous solution of NaHCO₃ and ethyl acetate were added, the organic layer was separated, and the aqueous phase was extracted twice with ethyl acetate. The combined organic extracts were washed with brine, dried over MgSO₄, filtered, and concentrated under reduced pressure to provide Intermediate 11-a as a beige solid.

### Step 3: Intermediate 11-b

To a solution of Intermediate 11-a (11.0 g, 13.9 mmol) in tert-butanol (147.0 ml) was added a 1.0 M solution of potassium tert-butoxide in tert-butanol (32.4 ml, 32.4 mmol). The reaction was stirred for 30 minutes at 80° C., then cooled to room temperature, and poured in 10% aqueous HCl. Ethyl acetate was added, the organic layer was separated, washed with brine, dried over MgSO₄, filtered, and concentrated under reduced pressure to provide Intermediate 11-b as a brown solid.

### Step 4: Intermediate 11-c

To a solution of Intermediate 11-b (11.0 g, 29.5 mmol) in ethyl acetate and stirred under nitrogen was added 10% Pd/C (1.25 g, 0.59 mmol). The reaction mixture was purged with H₂ and stirred for 3 hours under 1 atmosphere of hydrogen. The reaction was then filtered through celite, and the filtrate was concentrated in vacuo. Purification by silica gel chromatography provided Intermediate 11-c as a beige solid.

### Synthesis of Compound 2

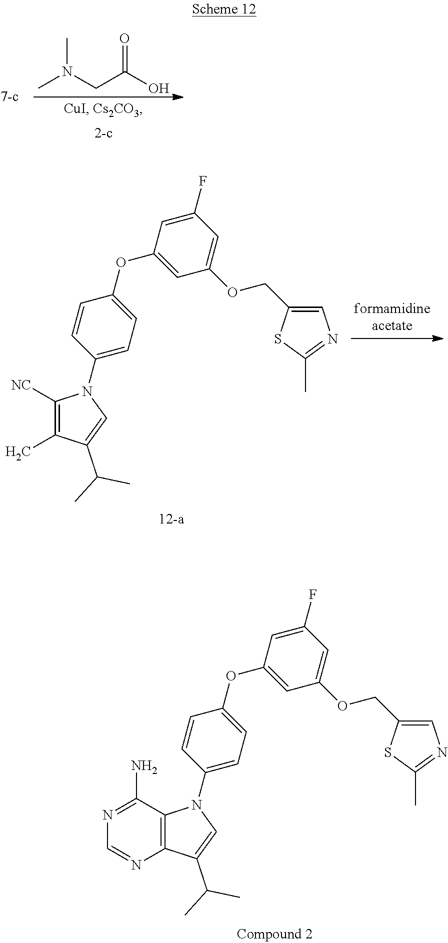

### Step 1: Intermediate 12-a

To a solution of Intermediate 7-c (375.0 mg, 1.3 mmol) in 1,4-dioxane (2.2 ml) was added Intermediate 2-c (601 mg, 1.9 mmol), N,N-dimethylglycine (342 mg, 3.3 mmol), copper (I) iodide (208 mg, 1.1 mmol), and cesium carbonate (2.1 g, 6.6 mmol). The reaction was heated at 110° C. overnight, and then cooled to room temperature, diluted with ethyl acetate, and filtered over celite. Volatiles were removed under reduced pressure. Purification by silica gel chromatography provided Intermediate 12-a as a beige foam.

### Step 2: Compound 2

To a solution of Intermediate 12-a (580 mg, 1.2 mmol) in methanol (2.5 ml) was added formamidine acetate (653 mg, 6.3 mmol), the reaction was stirred at reflux overnight, and then cooled to room temperature. Volatiles were removed under reduced pressure. Purification by reverse phase chromatography eluting with a 0.1% formic acid/methanol gradient provided Compound 2 as an off-white solid. MS (m/z) M+H=490.1

### Synthesis of Compound 3

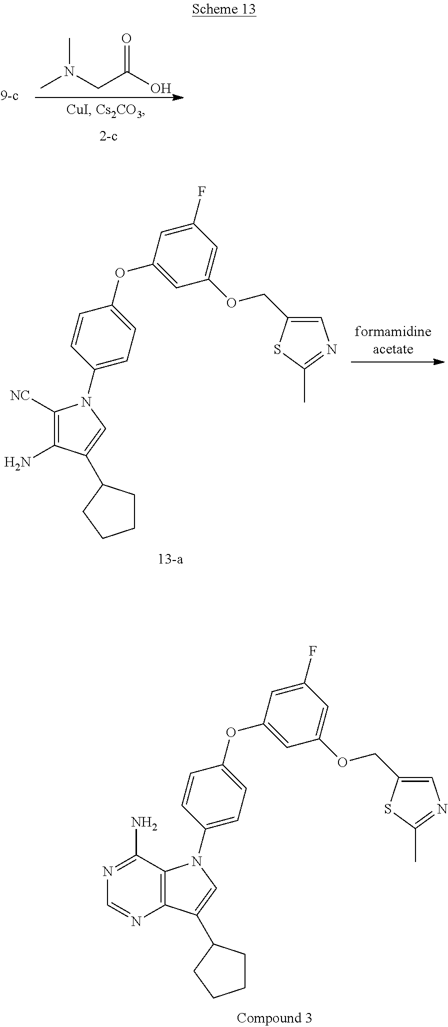

### Step 1: Intermediate 13-a

To a solution of Intermediate 9-c (400 mg, 1.5 mmol) in 1,4-dioxane (2.0 ml) was added Intermediate 2-c (500 mg, 1.6 mmol), N,N-dimethylglycine (309 mg, 2.9 mmol), copper (I) iodide (188 mg, 0.9 mmol), and cesium carbonate (2.1 g, 6.6 mmol). The reaction was heated at 110° C. overnight, and then cooled to room temperature, diluted with ethyl acetate, and filtered over celite. Volatiles were removed under reduced pressure. Purification by silica gel chromatography provided Intermediate 13-a as a beige foam.

### Step 2: Compound 3

To a solution of Intermediate 13-a (310 mg, 0.6 mmol) in methanol (1.3 ml) was added formamidine acetate (330 mg, 3.2 mmol) and the reaction was stirred at reflux overnight, and then cooled to room temperature. Volatiles were removed under reduced pressure. Purification by reverse phase chromatography eluting with a 0.1% formic acid/methanol gradient provided Compound 3 as a white solid. MS (m/z) M+H=516.2

### Synthesis of Compound 6

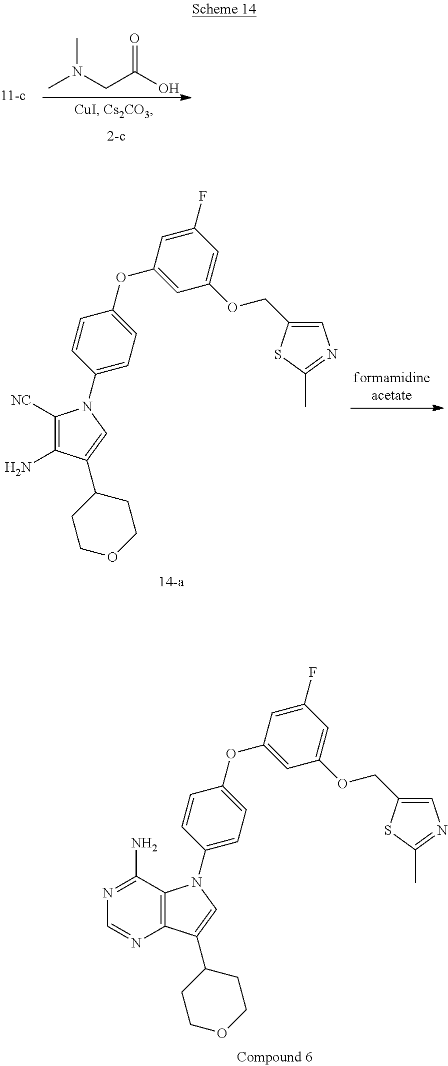

### Step 1: Intermediate 14-a

To a solution of Intermediate 11-c (400 mg, 1.4 mmol) in 1,4-dioxane (1.9 ml) was added intermediate 2-c (500 mg, 1.6 mmol), N,N-dimethylglycine (291 mg, 2.8 mmol), copper (I) iodide (177 mg, 0.9 mmol), and cesium carbonate (1.8 g, 5.6 mmol). The reaction was heated at 110° C. overnight, and then cooled to room temperature, diluted with ethyl acetate, and filtered over celite. Volatiles were removed under reduced pressure. Purification by silica gel chromatography provided Intermediate 14-a as a beige foam.

### Step 2: Compound 6

To a solution of Intermediate 14-a (630 mg, 1.2 mmol) in methanol (2.5 ml) was added formamidine acetate (650 mg, 6.2 mmol) and the reaction was stirred at reflux overnight, and then cooled to room temperature. Volatiles were removed under reduced pressure. Purification by silica gel chromatography provided Compound 6 as a beige solid. MS (m/z) M+H=532.2

### Synthesis of Compound 1

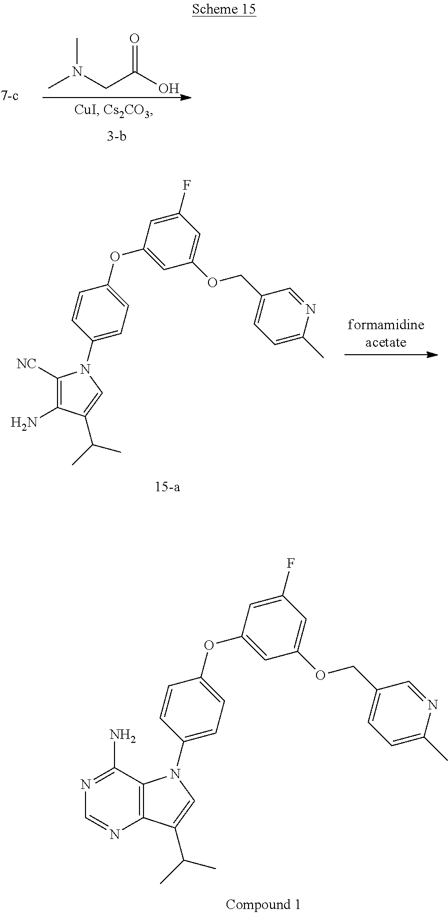

### Step 1: Intermediate 15-a

To a solution of Intermediate 7-c (407 mg, 1.7 mmol) in 1,4-dioxane (2.0 ml) was added Intermediate 3-b (600 mg, 2.0 mmol), N,N-dimethylglycine (348 mg, 3.4 mmol), copper (I) iodide (212 mg, 1.1 mmol), and cesium carbonate (2.2 g, 6.7 mmol). The reaction was heated at 110° C. overnight, and then cooled to room temperature, diluted with ethyl acetate, and filtered over celite. Volatiles were removed under reduced pressure. Purification by silica gel chromatography provided Intermediate 15-a as a beige foam.

### Step 2: Compound 1

To a solution of Intermediate 15-a (550 mg, 1.2 mmol) in methanol (2.4 ml) was added formamidine acetate (627 mg, 6.0 mmol), the reaction was stirred at reflux overnight, and then cooled to room temperature. Volatiles were removed under reduced pressure. Purification by reverse phase chromatography eluting with a 0.1% formic acid/methanol gradient provided Compound 1 as an off-white solid. MS (m/z) M+H=484.2

### Synthesis of Compound 4

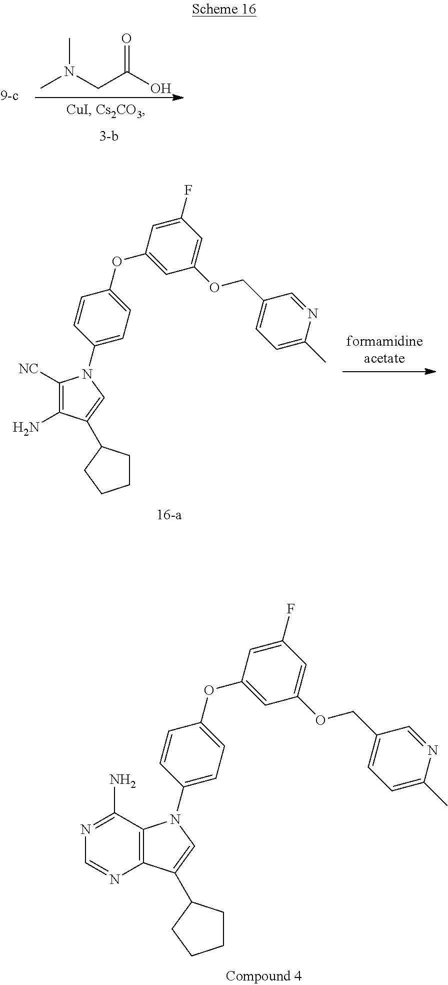

### Step 1: Intermediate 16-a

To a solution of Intermediate 9-c (400 mg, 1.5 mmol) in 1,4-dioxane (2.0 ml) was added intermediate 3-b (487 mg, 1.6 mmol), N,N-dimethylglycine (309 mg, 3.0 mmol), copper (I) iodide (188 mg, 0.9 mmol), and cesium carbonate (1.9 g, 6.0 mmol). The reaction was heated at 110° C. overnight, and then cooled to room temperature, diluted with ethyl acetate, and filtered over celite. Volatiles were removed under reduced pressure. Purification by silica gel chromatography provided Intermediate 16-a as a beige foam.

### Step 2: Compound 4

To a solution of Intermediate 16-a (550 mg, 1.0 mmol) in methanol (2.1 ml) was added formamidine acetate (539 mg, 5.2 mmol), the reaction was stirred at reflux overnight, and then cooled to room temperature. Volatiles were removed under reduced pressure. Purification by reverse phase chromatography eluting with a 0.1% formic acid/methanol gradient provided compound 4 as a white foam. 1N HCl was added to compound 4, a precipitate formed, and was collected by filtration, to provide Compound 4.2HCl as a beige solid MS (m/z) M+H=510.2

### Synthesis of Compound 5

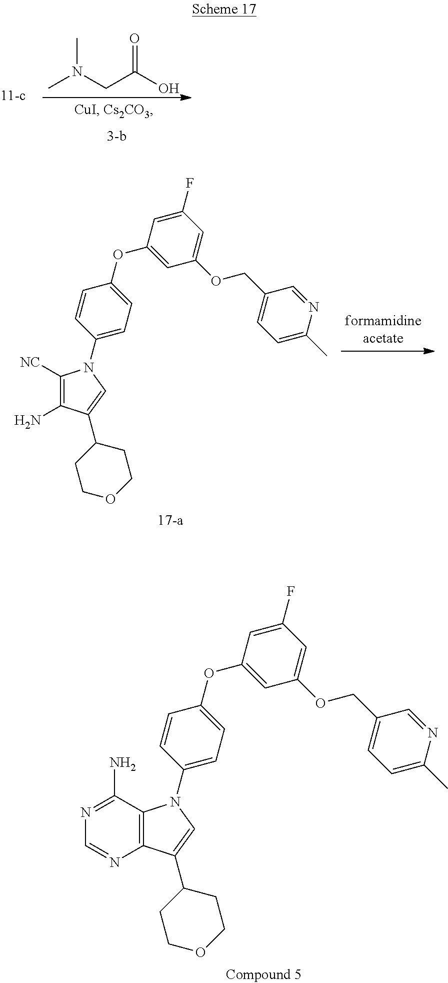

### Step 1: Intermediate 17-a

To a solution of Intermediate 11-c (400 mg, 1.4 mmol) in 1,4-dioxane (1.9 ml) was added Intermediate 3-b (460 mg, 1.5 mmol), N,N-dimethylglycine (291 mg, 2.8 mmol), copper (I) iodide (177 mg, 0.9 mmol), and cesium carbonate (1.9 g, 5.6 mmol). The reaction was heated in a sealed tube at 110° C. overnight, and then cooled to room temperature, diluted with ethyl acetate, and filtered over celite. Volatiles were removed under reduced pressure. Purification by silica gel chromatography provided Intermediate 17-a as a beige foam.

### Step 2: Compound 5

To a solution of Intermediate 17-a (520 mg, 1.0 mmol) in methanol (2.1 ml) was added formamidine acetate (543 mg, 5.2 mmol), the reaction was stirred at reflux overnight, and then cooled to room temperature. Volatiles were removed under reduced pressure. Purification by silica gel chromatography provided compound 5 as a white foam. 1N HCl was added to compound 5, a precipitate formed, and was collected by filtration to provide Compound 5.2HCl as a beige solid. MS (m/z) M+H=526.2

### Synthesis of Compound 9

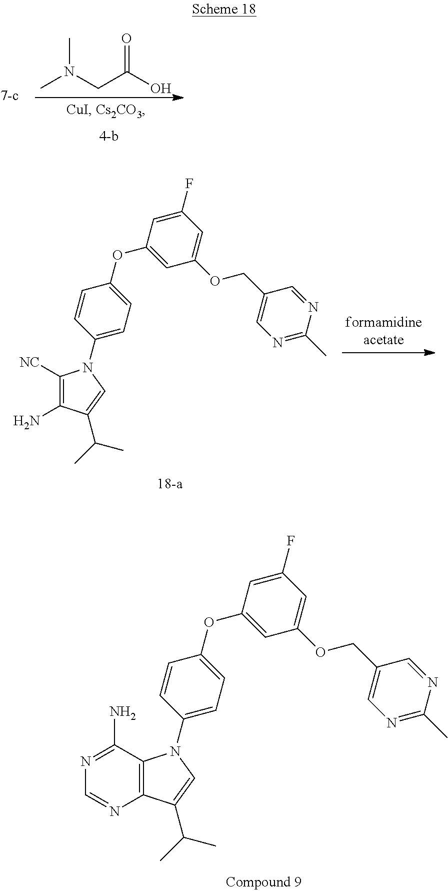

### Step 1: Intermediate 18-a

To a solution of Intermediate 7-c (300 mg, 1.2 mmol) in 1,4-dioxane (1.6 ml) was added Intermediate 4-b (443 mg, 1.5 mmol), N,N-dimethylglycine (256 mg, 2.5 mmol), copper (I) iodide (156 mg, 0.8 mmol), and cesium carbonate (1.6 g, 4.9 mmol). The reaction was heated at 110° C. overnight, and then cooled to room temperature, diluted with ethyl acetate, and filtered over celite. Volatiles were removed under reduced pressure. Purification by silica gel chromatography provided Intermediate 18-a as a beige foam.

### Step 2: Compound 9

To a solution of Intermediate 18-a (400 mg, 0.9 mmol) in methanol (8.7 ml) was added formamidine acetate (910 mg, 8.7 mmol), the reaction was stirred at reflux overnight, and then cooled to room temperature. Volatiles were removed under reduced pressure. Purification by reverse phase chromatography eluting with a 0.1N HCl/methanol gradient provided Compound 9.2HCl as a white solid. MS (m/z) M+H=485.2

### Synthesis of Compound 8

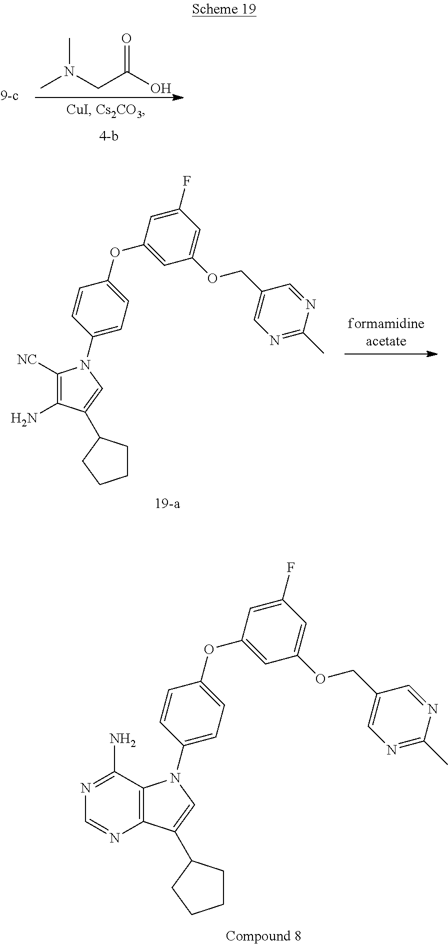

### Step 1: Intermediate 19-a

To a solution of Intermediate 9-c (300 mg, 1.1 mmol) in 1,4-dioxane (1.5 ml) was added Intermediate 4-b (367 mg, 1.2 mmol), N,N-dimethylglycine (231 mg, 2.2 mmol), copper (I) iodide (141 mg, 0.7 mmol), and cesium carbonate (1.5 g, 4.5 mmol). The reaction was heated at 110° C. overnight, and then cooled to room temperature, diluted with ethyl acetate, and filtered over celite. Volatiles were removed under reduced pressure. Purification by silica gel chromatography provided Intermediate 19-a as a beige foam

### Step 2: Compound 8

To a solution of Intermediate 19-a (564 mg, 1.2 mmol) in methanol (11.6 ml) was added formamidine acetate (1.2 mg, 11.6 mmol), the reaction was stirred at reflux overnight, and then cooled to room temperature. Volatiles were removed under reduced pressure. Purification by reverse phase chromatography eluting with a 0.1N HCl/methanol gradient provided Compound 8.2HCl as a white solid. MS (m/z) M+H=511.2

### Synthesis of Compound 7

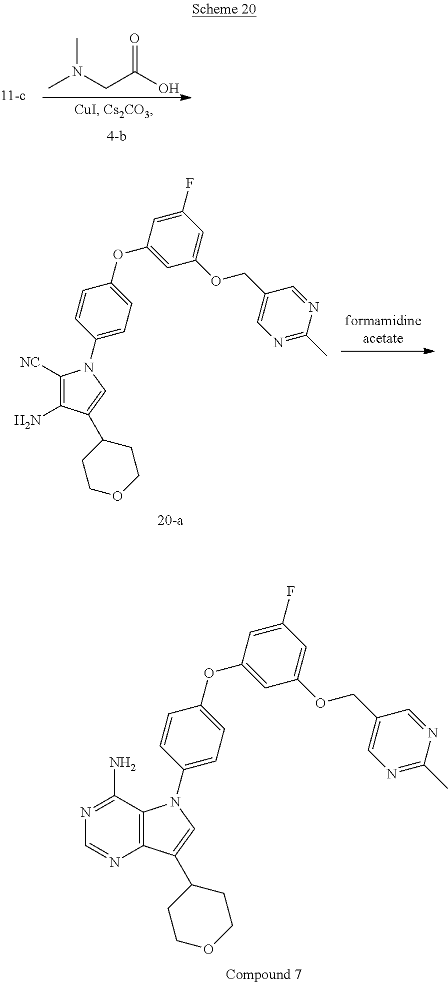

### Step 1: Intermediate 20-a

To a solution of Intermediate 11-c (300 mg, 1.1 mmol) in 1,4-dioxane (1.5 ml) was added Intermediate 4-b (346 mg, 1.2 mmol), N,N-dimethylglycine (218 mg, 2.2 mmol), copper (I) iodide (133 mg, 0.7 mmol) and cesium carbonate (1.4 g, 4.2 mmol). The reaction was heated at 110° C. overnight, and then cooled to room temperature, diluted with ethyl acetate, and filtered over celite. Volatiles were removed under reduced pressure. Purification by silica gel chromatography provided Intermediate 20-a as a beige foam.

### Step 2: Compound 7

To a solution of Intermediate 20-a (520 mg, 1.0 mmol) in methanol (10.4 ml) was added formamidine acetate (1.1 g, 10.4 mmol), the reaction was stirred at reflux overnight, and then cooled to room temperature. Volatiles were removed under reduced pressure. Purification by reverse phase chromatography eluting with a 0.1N HCl/methanol gradient provided Compound 7.2HCl as a beige solid. MS (m/z) M+H=527.2

### Synthesis of Intermediate 21-g

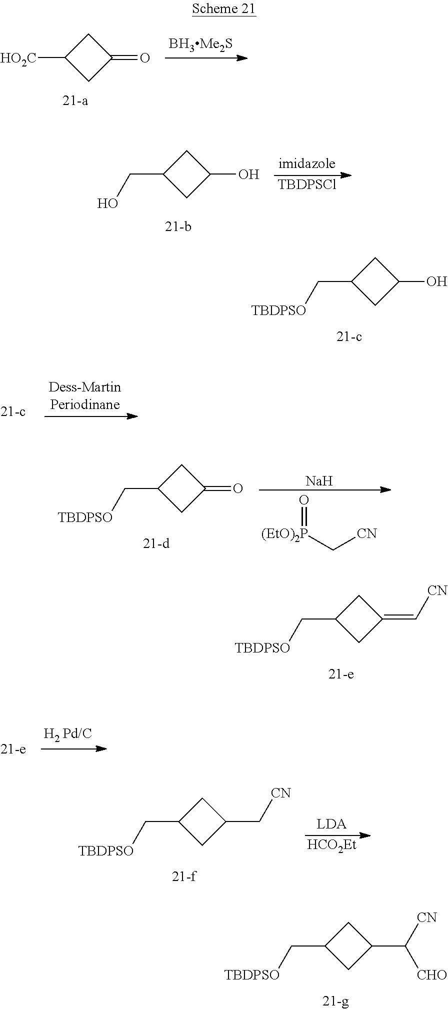

### Step 1: Intermediate 21-b

To a solution of 3-oxocyclobutanecarboxylic acid 21-a (6.2 g, 54.8 mmol) in THF (78 ml) cooled to −15° C. was slowly added BH₃.Me₂S (38.3 ml, 77.0 mmol), the reaction mixture was slowly warmed to room temperature, and stirred overnight. Methanol was slowly added and volatiles were removed under reduced pressure. Purification by silica gel chromatography provided Intermediate 21-b as a colorless oil.

### Step 2: Intermediate 21-c

To a solution of Intermediate 21-b (1.0 g, 9.8 mmol) in THF (49.0 ml) cooled to −10° C. were sequentially added imidazole (633 mg, 9.3 mmol) and tert-butyldiphenylsilyl chloride (1.4 g, 9.3 mmol), and the reaction was stirred at −10° C. for 30 minutes, and then room temperature overnight. Volatiles were removed under reduced pressure. Purification by silica gel chromatography provided Intermediate 21-c as a colorless oil.

### Step 3: Intermediate 21-d

To a solution of Intermediate 21-c (7.8 g, 22.9 mmol) in dichloromethane (229 ml) cooled to 0° C. were sequentially added sodium bicarbonate (19.3 g, 229.0 mmol) and Dess-Martin Periodinane (14.6 g, 34.4 mmol). The reaction mixture was warmed to room temperature and stirred for 2 hours. Volatiles were removed under reduced pressure. A saturated aqueous solution of NaHCO₃ and ethyl acetate were added to the residue, the organic layer was separated, washed with brine, dried over MgSO₄, filtered, and concentrated under reduced pressure to provide Intermediate 21-d as a yellow solid.

### Step 4: Intermediate 21-e

To a suspension of NaH (480 mg, 12.0 mmol) in diethyl ether (62 ml) cooled to 0° C. was added diethyl cyanomethylphosphonate (2.5 g, 14.2 mmol) dropwise followed by a solution of Intermediate 21-d (3.0 g, 8.9 mmol) in diethyl ether (62 ml). After the addition was completed the reaction was warmed to room temperature, and stirred overnight. Water and ethyl acetate were added, the organic layer was separated, washed with brine, dried over MgSO₄, filtered, and concentrated under reduced pressure. Purification by silica gel chromatography provided Intermediate 21-e as a colorless oil.

### Step 5: Intermediate 21-f

To a solution of Intermediate 21-e (2.0 g, 5.5 mmol) in ethanol stirred under nitrogen was added 10% Pd/C (1.2 g, 0.5 mmol). The reaction mixture was purged with H₂, and stirred overnight, under 1 atmosphere of hydrogen. The reaction was then filtered through celite, and the filtrate was concentrated in vacuo, to provide Intermediate 21-f as a beige oil.

### Step 6: Intermediate 21-g

To a solution of Intermediate 21-f (1.9 g, 5.2 mmol) in THF (1.7 ml) cooled to −78° C. was added drop wise a 2.0 M solution of LDA in THF (2.6 ml, 5.2 mmol). The solution was stirred for 10 minutes, and then added to a solution of ethyl formate (406 mg, 5.5 mmol) in THF (2.2 ml) cooled to −78° C. The reaction was stirred at −78° C. for 30 minutes, then slowly warmed to room temperature, and stirred overnight. The reaction was quenched by addition of 1N HCl until pH=3, and then extracted with ethyl acetate. The combined organic extracts were dried over MgSO₄, filtered, and concentrated under reduced pressure, to provide Intermediate 21-g as a yellow oil.

### Synthesis of Intermediate 22-c

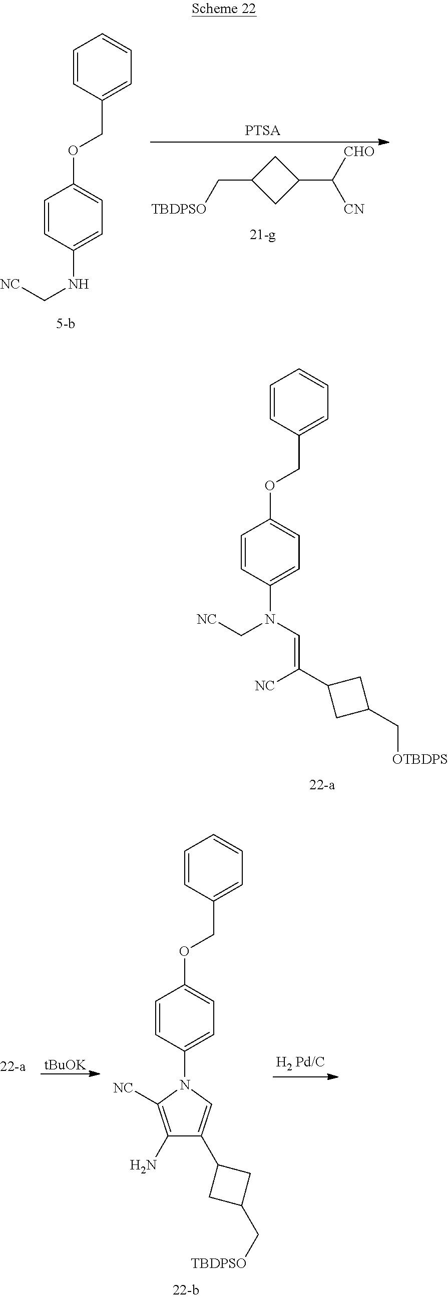

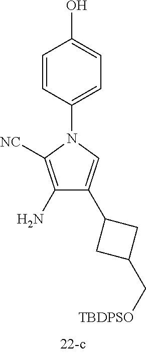

### Step 1: Intermediate 22-a

To a solution of Intermediate 5-b (1.4 g, 5.6 mmol) in toluene (20 ml), was added intermediate 21-g (2.0 g, 5.1 mmol) and PTSA (97 mg, 0.5 mmol). The reaction was stirred at reflux overnight using a Dean-Stark apparatus, and then cooled to room temperature. A saturated aqueous solution of NaHCO₃ and ethyl acetate were added, the organic layer was separated, and the aqueous phase was extracted twice with ethyl acetate. The combined organic extracts were washed with brine, dried over MgSO₄, filtered, and concentrated under reduced. Purification by silica gel chromatography provided Intermediate 22-a as a beige solid.

### Step 2: Intermediate 22-b

To a solution of Intermediate 22-a (630 mg, 1.0 mmol) in tert-butanol (5.0 ml) was added a 1.0 M solution of potassium tert-butoxide in tert-butanol (1.1 ml, 1.1 mmol). The reaction was stirred for 30 minutes at 80° C., then cooled to room temperature, and poured in a saturated aqueous solution of ammonium chloride. Ethyl acetate was added, the organic layer was separated, washed with brine, dried over MgSO₄, filtered, and concentrated under reduced pressure, to provide Intermediate 22-b as a brown solid.

### Step 3: Intermediate 22-c

To a solution of Intermediate 22-b (600 mg, 1.0 mmol) in ethyl acetate and stirred under nitrogen was added 10% Pd/C (209 mg, 0.1 mmol). The reaction mixture was purged with H₂, and stirred for 3 hours under 1 atmosphere of hydrogen. The reaction was then filtered through celite, and the filtrate was concentrated in vacuo. Purification by silica gel chromatography provided Intermediate 22-c as a beige solid.

### Synthesis of Compound 10

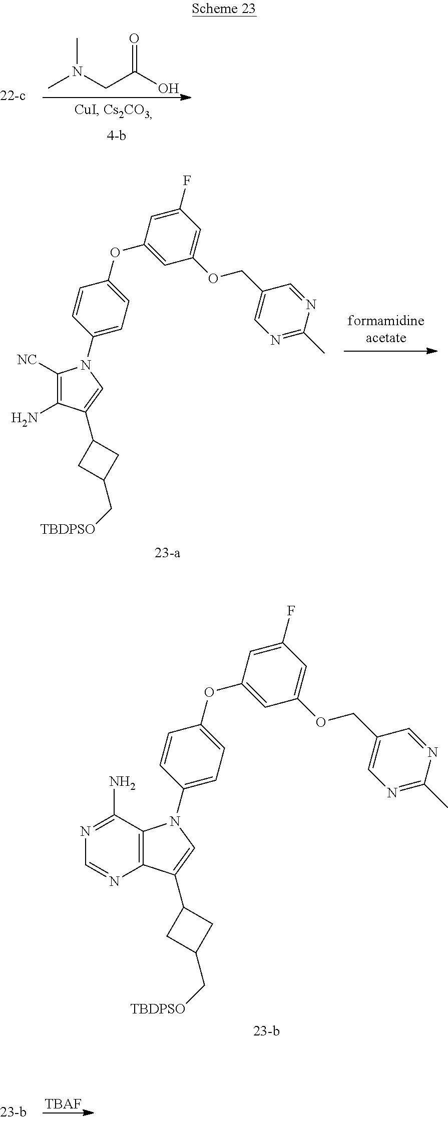

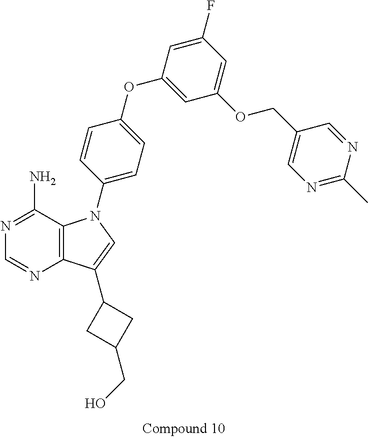 [SMILES: Cc1ncc(COc2cc(F)cc(Oc3ccc(-n4cc(C5CC(CO)C5)c5ncnc(N)c54)cc3)c2)cn1]

### Step 1: Intermediate 23-a

To a solution of Intermediate 22-c (188 mg, 0.4 mmol) in 1,4-dioxane (0.5 ml) was added Intermediate 4-b (118 mg, 0.4 mmol), N,N-dimethylglycine (74 mg, 0.7 mmol), copper (I) iodide (45 mg, 0.2 mmol), and cesium carbonate (470 mg, 1.4 mmol). The reaction was heated at 110° C. overnight, and then cooled to room temperature, diluted with ethyl acetate, and filtered over celite. Volatiles were removed under reduced pressure. Purification by silica gel chromatography provided Intermediate 23-a as a beige foam.

### Step 2: Intermediate 23-b

To a solution of Intermediate 23-a (222 mg, 0.3 mmol) in isopropanol (2.0 ml) was added formamidine acetate (1.0 g, 9.6 mmol), the reaction was stirred at reflux overnight, and then cooled to room temperature. A saturated aqueous solution of ammonium chloride and ethyl acetate were added, the organic layer was separated, washed with brine, dried over MgSO₄, filtered, and concentrated under reduced pressure, to provide Intermediate 23-b as a beige foam.

### Step 3: Compound 10

To a solution of Intermediate 23-b (110 mg, 0.1 mmol) in THF (2 mL) was added a 1.0 M solution of TBAF in THF (0.4 mL, 0.4 mmol) at room temperature, and the solution was then stirred for 2 days. Volatiles were removed under reduced pressure. Purification by reverse phase chromatography eluting with a 0.1% formic acid/methanol gradient provided Compound 10 (cis/trans mixture) as a white solid. MS (m/z) M+H=527.2

### Synthesis of Intermediate 24-d

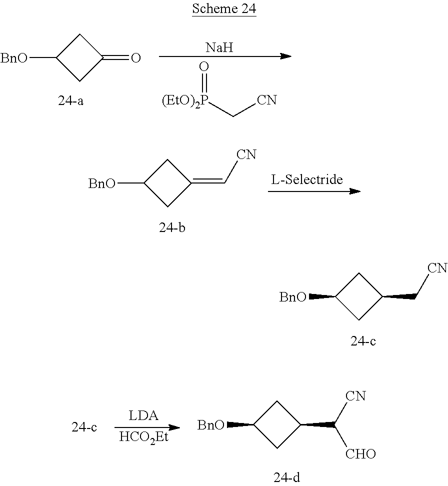

### Step 1: Intermediate 24-b

To a suspension of NaH (250 mg, 6.2 mmol) in THF (16 ml) cooled to 0° C. was added diethyl cyanomethylphosphonate (0.3 g, 7.4 mmol) drop wise followed by a solution of intermediate 24-a (1.0 g, 5.7 mmol) in THF (62 ml). After the addition was completed the reaction was warmed to room temperature and stirred overnight. Water and ethyl acetate were added, the organic layer was separated, washed with brine, dried over MgSO₄, filtered, and concentrated under reduced pressure. Purification by silica gel chromatography provided Intermediate 24-b as a colorless oil.

### Step 2: Intermediate 24-c

To a solution of Intermediate 24-b (940 mg, 4.7 mmol) in ethanol (23.5 ml) cooled to −78° C. was added a 1.0 M solution of L-Selectride in THF (5.2 ml, 5.2 mmol) and the reaction was stirred at −78° C. until completion. Brine (5.2 mL), 1.0 M aqueous solution of NaOH (5.2 mL), and 30 percent aqueous H₂O₂ (2.2 mL) were successfully added, and the mixture was stirred at room temperature for 30 minutes. Na₂SO₃ was then added and the mixture was extracted twice with ethyl acetate. The combined organic extracts were washed with brine, dried over MgSO₄, filtered and concentrated under reduced pressure, to provide Intermediate 24-c as a beige oil.

### Step 3: Intermediate 24-d

To a solution of Intermediate 24-c (860 mg, 4.3 mmol) in THF (19.0 ml) cooled to −78° C. was added drop wise a 2.0 M solution of LDA in THF (2.1 ml, 4.2 mmol). The solution was stirred for 10 minutes, and then added to a solution of ethyl formate (380 mg, 5.1 mmol) in THF (2.3 ml) cooled to −78° C. The reaction was stirred at −78° C. for 30 minutes, then slowly warmed to room temperature, and stirred overnight. The reaction was quenched by addition of 1N HCl until pH=3, and then extracted with ethyl acetate. The combined organic extracts were dried over MgSO₄, filtered, and concentrated under reduced pressure, to provide Intermediate 24-d as a yellow oil.

### Synthesis of Intermediate 25-c

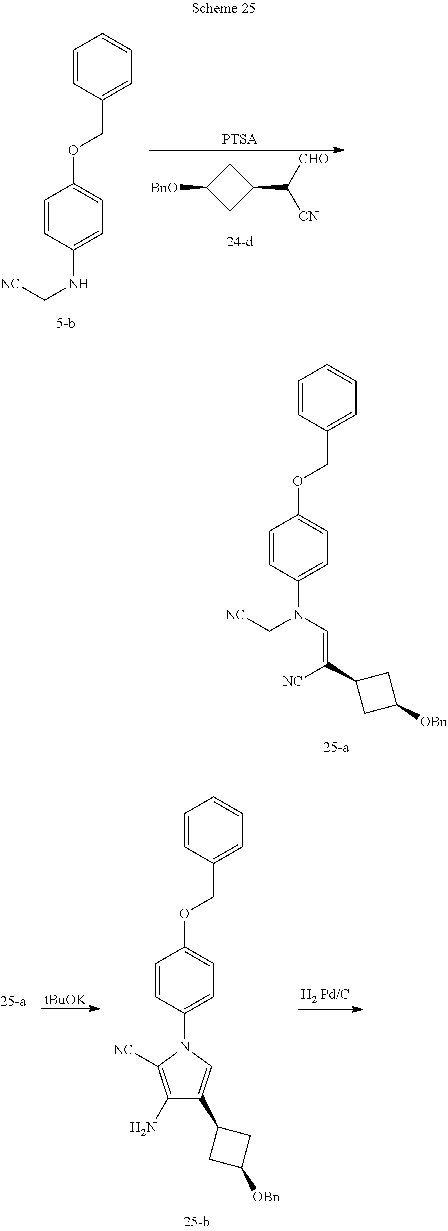 [SMILES: N#CCNc1ccc(OCc2ccccc2)cc1 ; N#CC(C=O)[C@H]1C[C@@H](OCc2ccccc2)C1 ; "tBuOK" ; N#CCN(/C=C(\C#N)[C@H]1C[C@@H](OCc2ccccc2)C1)c1ccc(OCc2ccccc2)cc1 ; N#Cc1c(N)c([C@H]2C[C@@H](OCc3ccccc3)C2)cn1-c1ccc(OCc2ccccc2)cc1]

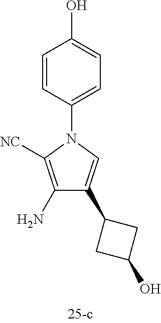 [SMILES: N#Cc1c(N)c([C@H]2C[C@@H](O)C2)cn1-c1ccc(O)cc1]

### Step 1: Intermediate 25-a

To a solution of Intermediate 5-b (391 mg, 1.6 mmol) in toluene (20 ml), was added Intermediate 24-d (342 mg, 1.5 mmol), and PTSA (28 mg, 0.1 mmol). The reaction was stirred at reflux overnight using a Dean-Stark apparatus, then cooled to room temperature. A saturated aqueous solution of NaHCO₃ and ethyl acetate were added, the organic layer was separated, and the aqueous phase was extracted twice with ethyl acetate. The combined organic extracts were washed with brine, dried over MgSO₄, filtered, and concentrated under reduced. Purification by silica gel chromatography provided Intermediate 25-a as a beige solid.

### Step 2: Intermediate 25-b

To a solution of Intermediate 25-a (240 mg, 0.5 mmol) in tert-butanol (2.6 ml) was added a 1.0 M solution of potassium tert-butoxide in tert-butanol (590 μl, 0.59 mmol). The reaction was stirred for 30 minutes at 80° C., then cooled to room temperature, and poured in a saturated aqueous solution of ammonium chloride. Ethyl acetate was added, the organic layer was separated, washed with brine, dried over MgSO₄, filtered, and concentrated under reduced pressure. Purification by silica gel chromatography provided Intermediate 25-b as a beige solid.

### Step 3: Intermediate 25-c

To a solution of Intermediate 25-b (115 mg, 0.2 mmol) in methanol, containing 2 drops of 37% aqueous HCl, and stirred under nitrogen was added 10% Pd/C (54 mg, 0.02 mmol). The reaction mixture was purged with H₂ and stirred overnight under 1 atmosphere of hydrogen. The reaction was then filtered through celite, and the filtrate was concentrated in vacuo to provide Intermediate 25-c as a beige solid.

### Synthesis of Compound 11

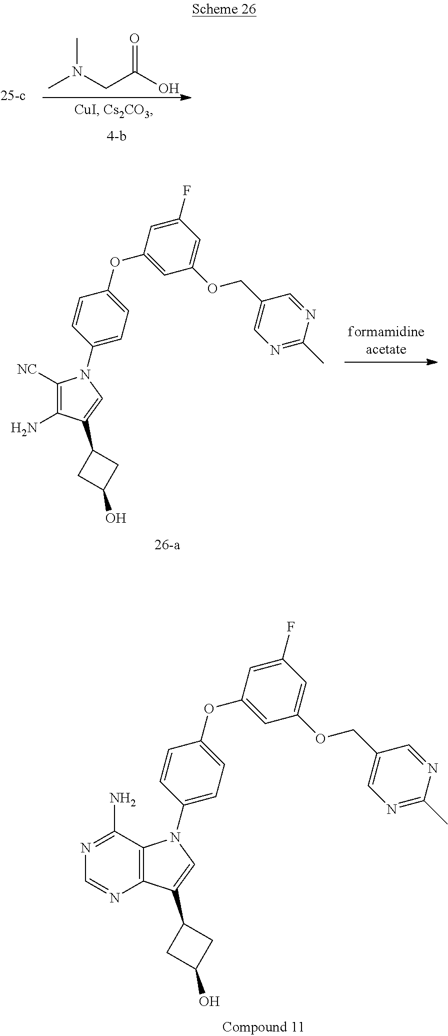

### Step 1: Intermediate 26-a

To a solution of Intermediate 25-c (70 mg, 0.3 mmol) in 1,4-dioxane (0.4 ml) and NMP (0.1 ml) was added Intermediate 4-b (93 mg, 0.3 mmol), N,N-dimethylglycine (54 mg, 0.5 mmol), copper (I) iodide (33 mg, 0.2 mmol), and cesium carbonate (339 mg, 1.0 mmol). The reaction was heated at 110° C. overnight, and then cooled to room temperature, diluted with ethyl acetate, and filtered over celite. Volatiles were removed under reduced pressure. Purification by silica gel chromatography provided Intermediate 26-a as a beige foam.

### Step 2: Compound 11

To a solution of Intermediate 26-a (11 mg, 0.02 mmol) in isopropanol (2.0 ml) was added formamidine acetate (100 mg, 0.9 mmol), the reaction was stirred at reflux overnight, and then cooled to room temperature. A saturated aqueous solution of ammonium chloride and ethyl acetate were added, the organic layer was separated, washed with brine, dried over MgSO₄, filtered, and concentrated under reduced pressure. Purification by reverse phase chromatography eluting with a 0.1% formic acid/methanol gradient provided Compound 10 (cis/trans mixture) as a white solid. MS (m/z) M+H=513.2.

TABLE1
Example Compounds of Formula I

| Compound | Structure | MS (m/z) |
|---|---|---|
| 1 | 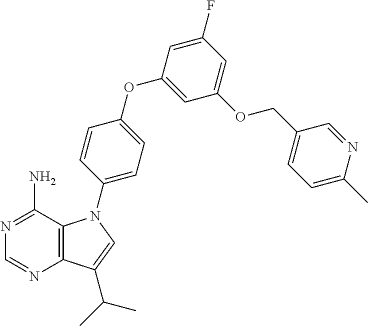 [SMILES: Cc1ccc(COc2cc(F)cc(Oc3ccc(-n4cc(C(C)C)c5ncnc(N)c54)cc3)c2)cn1] | [M + H]⁺ = 484.2; |
| 2 | 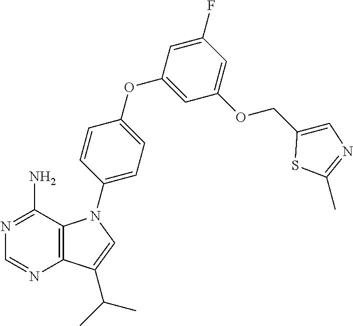 [SMILES: Cc1ncc(COc2cc(F)cc(Oc3ccc(-n4cc(C(C)C)c5ncnc(N)c54)cc3)c2)s1] | [M + H]⁺ = 490.1; |
| 3 | 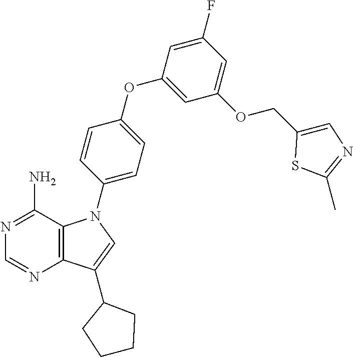 [SMILES: Cc1ncc(COc2cc(F)cc(Oc3ccc(-n4cc(C5CCCC5)c5ncnc(N)c54)cc3)c2)s1] | [M + H]⁺ = 516.2; |
| 4 | 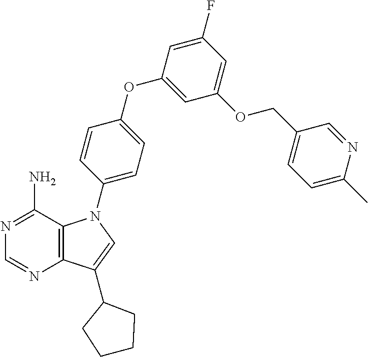 [SMILES: Cc1ccc(COc2cc(F)cc(Oc3ccc(-n4cc(C5CCCC5)c5ncnc(N)c54)cc3)c2)cn1] | [M + H]⁺ = 510.2; |
| 5 | 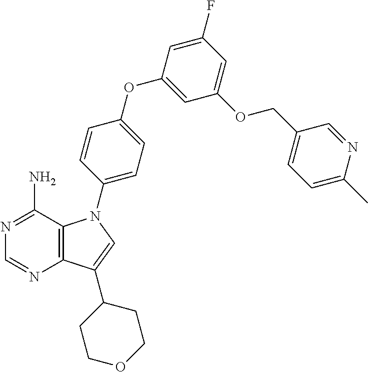 [SMILES: Cc1ccc(COc2cc(F)cc(Oc3ccc(-n4cc(C5CCOCC5)c5ncnc(N)c54)cc3)c2)cn1] | [M + H]⁺ = 526.2; |
| 6 | 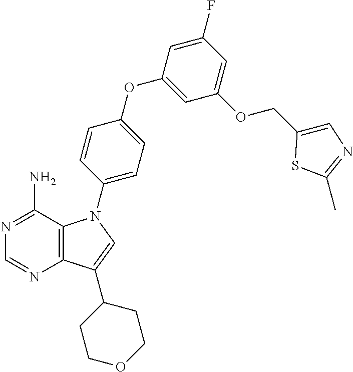 [SMILES: Cc1ncc(COc2cc(F)cc(Oc3ccc(-n4cc(C5CCOCC5)c5ncnc(N)c54)cc3)c2)s1] | [M + H]⁺ = 532.2; |
| 7 | 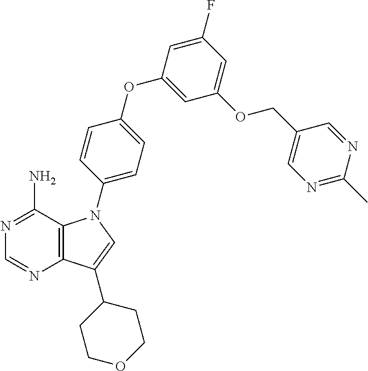 [SMILES: Cc1ncc(COc2cc(F)cc(Oc3ccc(-n4cc(C5CCOCC5)c5ncnc(N)c54)cc3)c2)cn1] | [M + H]⁺ = 527.2; |
| 8 | 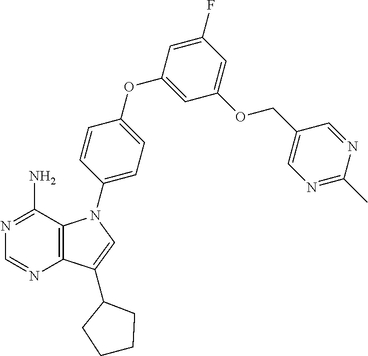 [SMILES: Cc1ncc(COc2cc(F)cc(Oc3ccc(-n4cc(C5CCCC5)c5ncnc(N)c54)cc3)c2)cn1] | [M + H]⁺ = 511.2; |
| 9 | 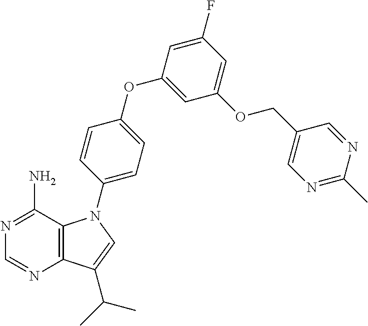 [SMILES: Cc1ncc(COc2cc(F)cc(Oc3ccc(-n4cc(C(C)C)c5ncnc(N)c54)cc3)c2)cn1] | [M + H]⁺ = 485.2; |
| 10 | 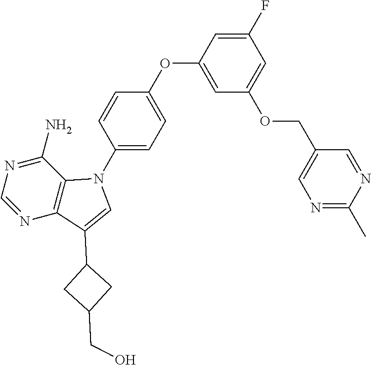 [SMILES: Cc1ncc(COc2cc(F)cc(Oc3ccc(-n4cc(C5CC(CO)C5)c5ncnc(N)c54)cc3)c2)cn1] | [M + H]⁺ = 527.2, or |
| 11 | 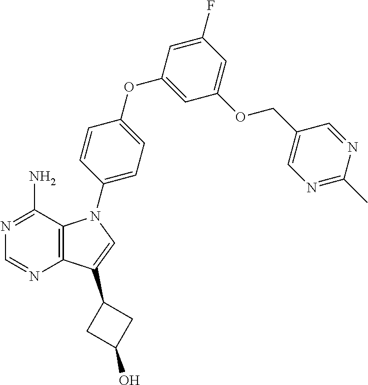 [SMILES: Cc1ncc(COc2cc(F)cc(Oc3ccc(-n4cc([C@H]5C[C@@H](O)C5)c5ncnc(N)c54)cc3)c2)cn1] | [M + H]⁺ = 513.2. |

### Biological Assays

Assays for determining kinase activity are described in more details in the accompanying examples.

### Kinase Inhibition

### Btk Kinase Inhibition Assays

### Method A

Fluorescence polarization-based kinase assays were performed in 384 well-plate format using histidine tagged recombinant human full-length Bruton Agammaglobulinemia Tyrosine Kinase (Btk), and a modified protocol of the KinEASE™ FP Fluorescein Green Assay supplied from Millipore®. Kinase reaction were performed at room temperature for 60 minutes, in presence of 250 μM substrate, 10 μM ATP, and variable test article concentrations. The reaction was stopped with EDTA/kinease detection reagents. Phosphorylation of the substrate peptide was detected by fluorescence polarization, measured with a Tecan 500 instrument. From the dose-response curve obtained, the IC₅₀ was calculated using Graph Pad Prisms®, using a non linear fit curve. The Km for ATP on each enzyme was experimentally determined and the Ki values calculated using the Cheng-Prusoff equation (see: Cheng Y, Prusoff W H. (1973) Relationship between the inhibition constant (K1), and the concentration of inhibitor which causes 50% inhibition (I₅₀) of an enzymatic reaction”. Biochem Pharmacol 22 (23): 3099-108).

kᵢ values are reported in Tables 2a and 2b:

TABLE 2a
Inhibition of Btk

| Compound | Ki (nM) |
|---|---|
| 1 | a |
| 2 | a |
| 5 | a |
| 6 | a |
| 7 | a |
| 9 | a |

a - Ki < 100 nM;
b - 100 nM < Ki < 1000 nM,
c - ki > 1000 nM

### Method B

In vitro potency of selected compound was defined against human BTK kinase (hBTK) using KinaseProfiler radiometric protein kinase assays performed at Eurofins Pharma Discovery Services UK Limited.

hBTK kinase is diluted in buffer and all compounds were prepared to 50× final assay concentration in 100% DMSO. This working stock of the compound was added to the assay well as the first component in the reaction, followed by the remaining components as detailed in the assay protocol listed above. The reaction was initiated by the addition of the MgATP mix. The kinase reaction was performed at room temperature for 40 minutes, in presence of 250 μM substrate, 10 mM MgAcetate, [γ-33P-ATP] (specific activity approx. 500 cpm/pmol, concentration as required) and variable test article concentrations. The ATP concentrations in the assays were with 15 μM of the apparent. The reaction was stopped by the addition of 3% phosphoric acid solution. 10 μL of the reaction is then spotted onto a P30 filtermat and washed three times for 5 minutes in 75 mM phosphoric acid and once in methanol, prior to drying, and scintillation counting. In addition positive control wells contain all components of the reaction, except the compound of interest; however, DMSO (at a final concentration of 2%) were included in these wells to control for solvent effects, as well as blank wells contain all components of the reaction, with a reference inhibitor replacing the compound of interest. This abolishes kinase activity and establishes the base-line (0% kinase activity remaining). The potency of each compound was reported by estimating the EC₅₀.

TABLE 2b
Inhibition of Btk

| Compound | EC₅₀ (nM) |
|---|---|
| 11 | a |

a - EC₅₀ < 100 nM,
b - 100 nM < EC₅₀ < 1000 nM,
c - EC₅₀ > 1000 nM

### Cellular Assay

### Splenic Cell Proliferation Assay

Proliferation of splenocytes in response to anti-IgM can be blocked by inhibition of Btk. Splenocytes were obtained from 6 week old male CD1 mice (Charles River Laboratories Inc.). Mouse spleens were manually disrupted in PBS and filtered using a 70 um cell strainer followed by ammonium chloride red blood cell lysis. Cells were washed, resuspended in Splenocyte Medium (HyClone RPMI supplemented with 10% heat-inactivated FBS, 0.5× non-essential amino acids, 10 mM HEPES, 50 uM beta mercaptoethanol), and incubated at 37° C., 5% CO₂ for 2 h, to remove adherent cells. Suspension cells were seeded in 96 well plates, at 50,000 cells per well, and incubated at 37° C., 5% CO₂ for 1 h. Splenocytes were pre-treated in triplicate with 10,000 nM curves of Formula I, compounds for 1 h, followed by stimulation of cell proliferation with 2.5 ug/ml anti-IgM F(ab′)₂ (Jackson Immuno Research) for 72 h. Cell proliferation was measured by Cell Titer-Glo Luminescent Assay (Promega). EC₅₀ values (50% proliferation in the presence of compound, as compared to vehicle treated controls) were calculated from dose response compound curves using GraphPad Prism Software.

EC₅₀ values are reported in Table 3:

TABLE 3
Inhibition of splenic cell proliferation

| Compound | EC₅₀ (nM) |
|---|---|
| 1 | a |
| 2 | a |
| 3 | a |
| 4 | a |
| 5 | a |
| 6 | a |
| 7 | a |
| 8 | a |
| 9 | a |
| 10 | a |
| 11 | a |

a - EC₅₀ < 100 nM;
b - 100 nM < EC₅₀ < 1000 nM,
c - EC₅₀ > 1000 nM

1. A compound of Formula I: 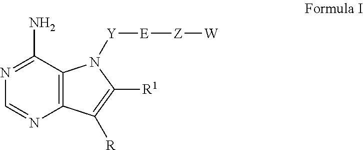
or pharmaceutically acceptable salt, solvate, solvate of salt, stereoisomer, tautomer, isotope, prodrug, complex or biologically active metabolite thereof,
wherein R is selected from the group consisting of:
1) hydrogen,
2) alkyl,
3) heteroalkyl,
4) carbocyclyl,
5) heterocyclyl,
6) aryl, or
7) heteroaryl,
wherein the alkyl, heteroalkyl, carbocyclyl, heterocycyl, aryl, or heteroaryl are optionally substituted;
R¹ is selected from the group consisting of:
1) hydrogen,
2) alkyl,
3) heteroalkyl,
4) carbocyclyl,
5) heterocyclyl, or
6) halogen,
wherein the alkyl, heteroalkyl, carbocyclyl, or heterocyclyl are optionally substituted;
Y is 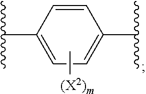
E is oxygen;
Z is 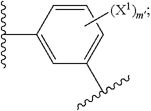
W is selected from:
1) —OCH₂R², or
2) —CH₂OR²,
wherein Y-E-Z-W is 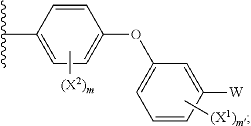
R² is selected from substituted or unsubstituted aryl, substituted or unsubstituted heteroaryl;
X¹ and X² are independently selected from hydrogen or halogen;
m is an integer from 0 to 4, or
m′ is an integer from 0 to 4.

2. The compound according to claim 1, wherein R is selected from the group consisting of:

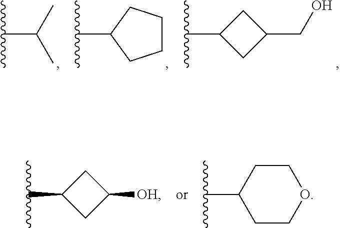

3. The compound according to claim 1, wherein R¹ is hydrogen.

4. The compound according to claim 1, wherein Z is

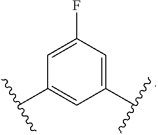 [SMILES: *c1cc(*)cc(F)c1]

5. The compound according to claim 1, wherein Y is

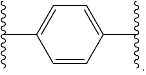 [SMILES: *c1ccc(*)cc1]

6. The compound according to claim 1, wherein W is selected from the group consisting of:

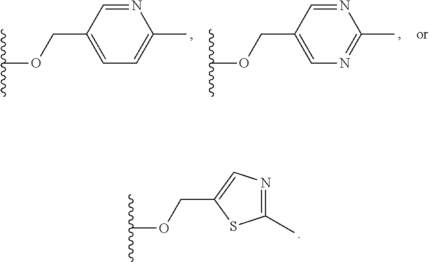

7. A compound of Formula II: 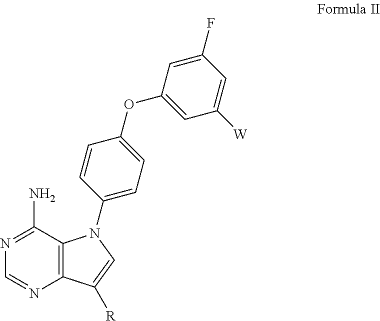
or a pharmaceutically acceptable salts, solvates, solvates of salts, stereoisomers, tautomers, isotopes, prodrugs, complexes or biologically active metabolites thereof, wherein R is selected from the group consisting of:
1) hydrogen,
2) alkyl,
3) heteroalkyl,
4) carbocyclyl,
5) heterocyclyl,
6) aryl, or
7) heteroaryl,
wherein the alkyl, heteroalkyl, carbocyclyl, heterocyclyl, aryl, or heteroaryl are optionally substituted; and
W is selected from the group consisting of: —OCH₂R², or —CH₂OR²,
wherein R² is selected from substituted or unsubstituted aryl, substituted or unsubstituted heteroaryl.

8. The compound according to claim 7, wherein R is:

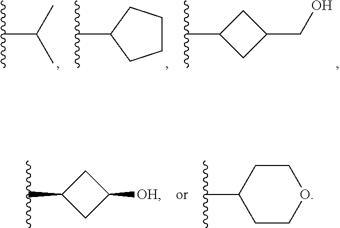

9. The compound according to claim 7, wherein W is:

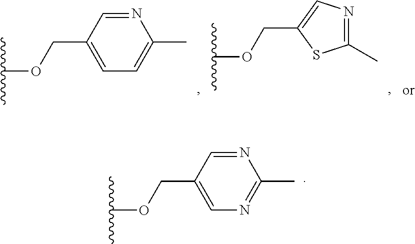

10. A compound selected from the group consisting of:

| Com- | |
|---|---|
| pound | Structure |
| 1 | 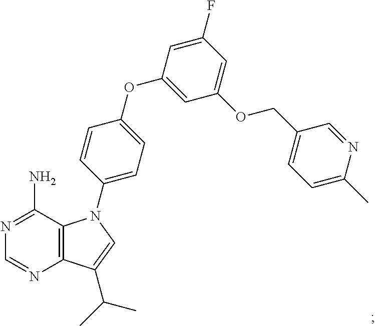 [SMILES: Cc1ccc(COc2cc(F)cc(Oc3ccc(-n4cc(C(C)C)c5ncnc(N)c54)cc3)c2)cn1] |
| 2 | 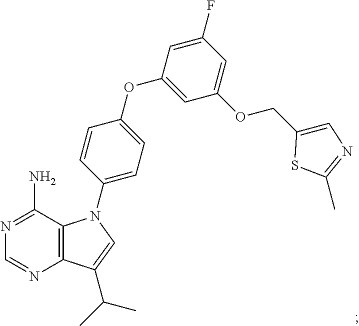 [SMILES: Cc1ncc(COc2cc(F)cc(Oc3ccc(-n4cc(C(C)C)c5ncnc(N)c54)cc3)c2)s1] |
| 3 | 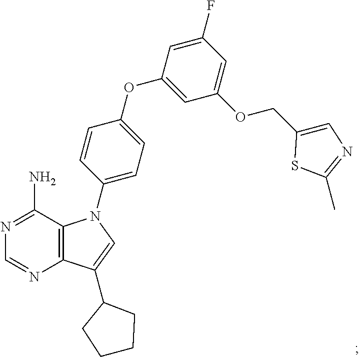 [SMILES: Cc1ncc(COc2cc(F)cc(Oc3ccc(-n4cc(C5CCCC5)c5ncnc(N)c54)cc3)c2)s1] |
| 4 | 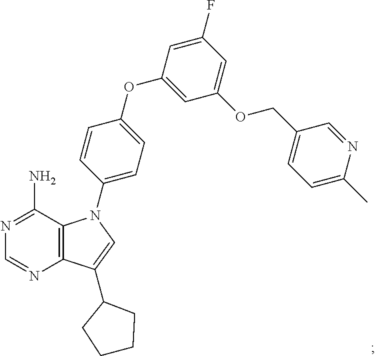 [SMILES: Cc1ccc(COc2cc(F)cc(Oc3ccc(-n4cc(C5CCCC5)c5ncnc(N)c54)cc3)c2)cn1] |
| 5 | 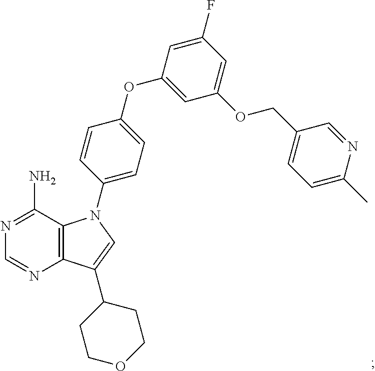 [SMILES: Cc1ccc(COc2cc(F)cc(Oc3ccc(-n4cc(C5CCOCC5)c5ncnc(N)c54)cc3)c2)cn1] |
| 6 | 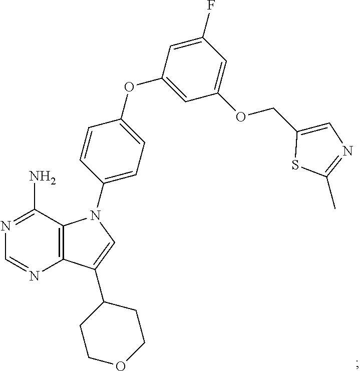 [SMILES: Cc1ncc(COc2cc(F)cc(Oc3ccc(-n4cc(C5CCOCC5)c5ncnc(N)c54)cc3)c2)s1] |
| 7 | 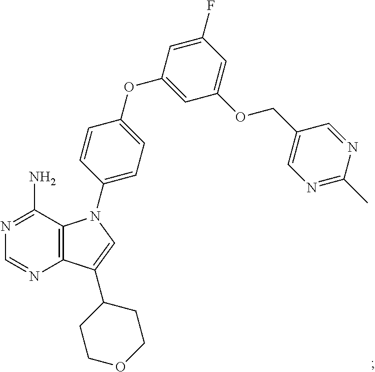 [SMILES: Cc1ncc(COc2cc(F)cc(Oc3ccc(-n4cc(C5CCOCC5)c5ncnc(N)c54)cc3)c2)cn1] |
| 8 | 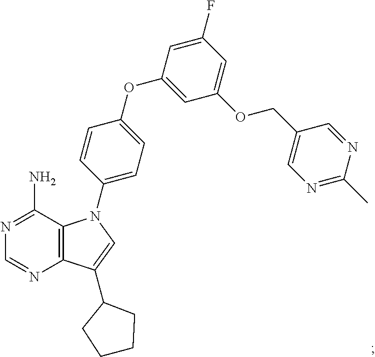 [SMILES: Cc1ncc(COc2cc(F)cc(Oc3ccc(-n4cc(C5CCCC5)c5ncnc(N)c54)cc3)c2)cn1] |
| 9 | 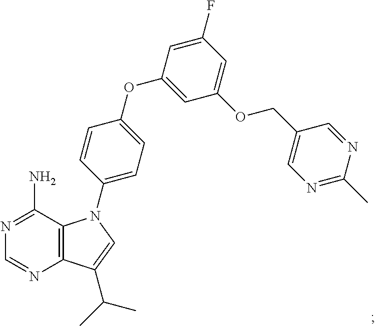 [SMILES: Cc1ncc(COc2cc(F)cc(Oc3ccc(-n4cc(C(C)C)c5ncnc(N)c54)cc3)c2)cn1] |
| 10 | 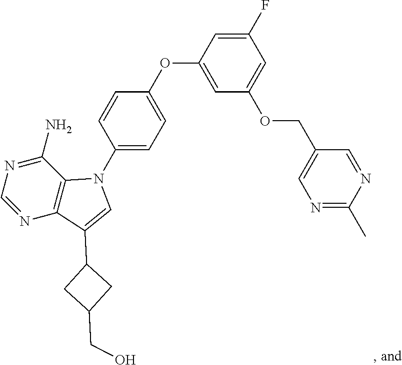 [SMILES: Cc1ncc(COc2cc(F)cc(Oc3ccc(-n4cc(C5CC(CO)C5)c5ncnc(N)c54)cc3)c2)cn1] |
| 11 | 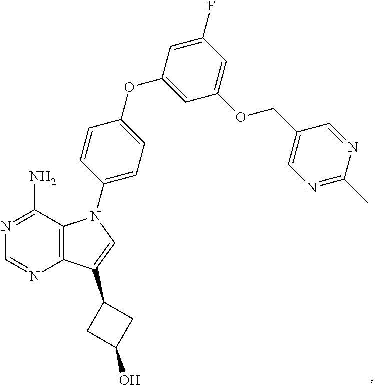 [SMILES: Cc1ncc(COc2cc(F)cc(Oc3ccc(-n4cc([C@H]5C[C@@H](O)C5)c5ncnc(N)c54)cc3)c2)cn1] |

or a pharmaceutically acceptable salt, solvate, solvate of salt, stereoisomer, tautomer, isotope, prodrug, complex or biologically active metabolite thereof.

11. A process for preparing a compound of claim 1, wherein the process comprises the following steps: 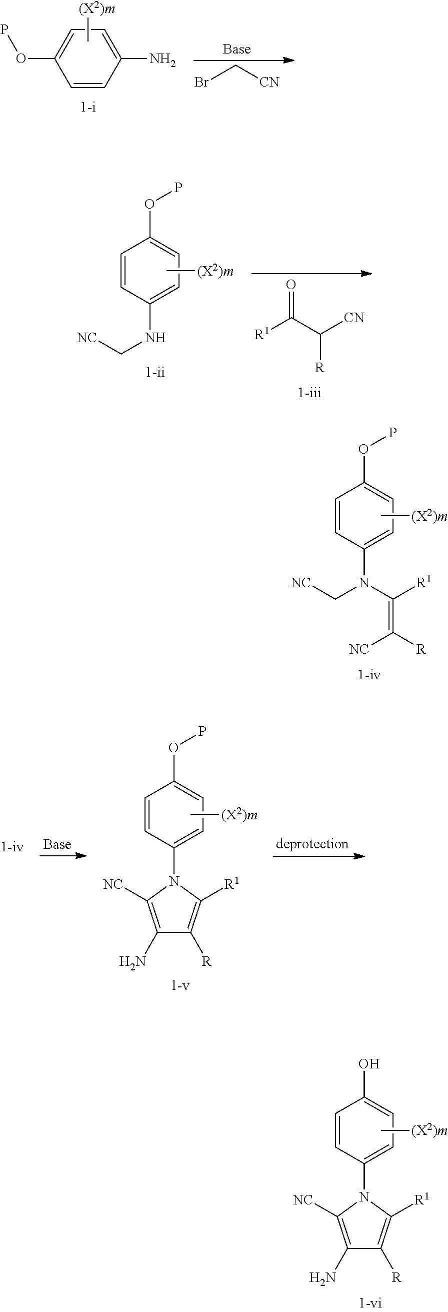 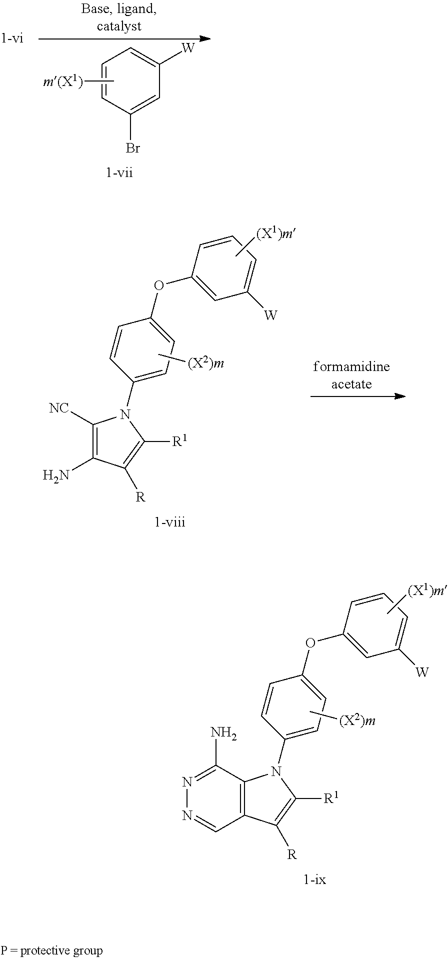
wherein P is a protective group, R, R¹, X, X¹, m, m′, and W as defined in claim 1.

12. A compound of any one of claims 1 to 10, or a pharmaceutically acceptable salt, solvate, solvate of salt, stereoisomer, tautomer, isotope, prodrug, complex or biologically active metabolite thereof, for use in therapy.

13. The compound according to any one of claims 1 to 10, for use in the treatment of a proliferative, inflammatory, infectious or autoimmune diseases.

14. The compound for use according to claim 13, wherein the proliferative disease is cancer.

15. The compound for use according to claim 13, wherein the proliferative disease is an autoimmune disease, inflammatory disorder, or state characterized by inflammation or cell proliferation.

16. The compound according to any one of claims 1 to 10, for use in the treatment of a subject suffering from a protein kinase mediated disease, disorder, or condition in which Tec kinase family member activity is implicated.

17. The compound according to any one of claims 1 to 10, for use in the treatment of a subject suffering from a protein kinase mediated disease, disorder, or condition in which Src kinase family member activity is implicated.

18. The compound according to any one of claims 1 to 10, for use in the treatment of a subject suffering from a protein kinase mediated disease, disorder, or condition in which Btk kinase family member activity is implicated.

19. The compound according to any one of claims 1 to 10 or 12 to 18, for use in the treatment of a proliferative disorder, disease, or condition, in combination with an agent selected from: an estrogen receptor modulator; an androgen receptor modulator; a retinoid receptor modulator; a cytotoxic agent; an anti-proliferative agent comprises adriamycin, dexamethasone, vincristine, cyclophosphamide, fluorouracil, topotecan, taxol, interferons, or platinum derivatives; an anti-inflammatory agent comprises corticosteroids, TNF blockers, IL-1 RA, azathioprine, cyclophosphamide, or sulfasalazine; a prenyl-protein transferase inhibitor; an HMG-CoA reductase inhibitor; an HIV protease inhibitor; a reverse transcriptase inhibitor; an angiogenesis inhibitor comprises sorafenib, sunitinib, pazopanib, or everolimus; an immunomodulatory, or immunosuppressive agents comprises: cyclosporin, tacrolimus, rapamycin, mycophenolate mofetil, interferons, corticosteroids, cyclophophamide, azathioprine, or sulfasalazine; a PPAR-γ agonist comprising thiazolidinediones; a PPAR-δ agonist; an inhibitor of inherent multidrug resistance; an agent for the treatment of anemia, comprising erythropoiesis-stimulating agents, vitamins, or iron supplements; an anti-emetic agent including 5-HT3 receptor antagonists, dopamine antagonists, NK1 receptor antagonist, H1 histamine receptor antagonists, cannabinoids, benzodiazepines, anticholinergic agents, or steroids; an agent for the treatment of neutropenia; an immunologic-enhancing agents; a proteasome inhibitors; an HDAC inhibitors; an inhibitor of the chemotrypsin-like activity in the proteasome; a E3 ligase inhibitors; a modulator of the immune system including interferon-alpha, Bacillus Calmette-Guerin (BCG), or ionizing radiation (UVB) that can induce the release of cytokines, interleukins, TNF, or induce release of death receptor ligands including TRAIL; a modulator of death receptors TRAIL, or TRAIL agonists including humanized antibodies HGS-ETR1, or HGS-ETR2; neurotrophic factors selected from cetylcholinesterase inhibitors, MAO inhibitors, interferons, anti-convulsants, ion channel blockers, or riluzole; anti-Parkinsonian agents comprising anticholinergic agents, or dopaminergic agents, including dopaminergic precursors, monoamine oxidase B inhibitors, COMT inhibitors, or dopamine receptor agonists; agents for treating cardiovascular disease comprises beta-blockers, ACE inhibitors, diuretics, nitrates, calcium channel blockers, or statins; agents for treating liver disease comprises corticosteroids, cholestyramine, or interferons; anti-viral agents, including nucleoside reverse transcriptase inhibitors, non-nucleoside reverse transcriptase inhibitors, protease inhibitors, integrase inhibitors, fusion inhibitors, chemokine receptor antagonists, polymerase inhibitors, viral proteins synthesis inhibitors, viral protein modification inhibitors, neuraminidase inhibitors, fusion or entry inhibitors; agents for treating blood disorders comprising corticosteroids, anti-leukemic agents, or growth factors; agents for treating immunodeficiency disorders comprising gamma globulin, adalimumab, etarnecept, or infliximab; a HMG-CoA reductase inhibitors including torvastatin, fluvastatin, lovastatin, pravastatin, rosuvastatin, simvastatin, or pitavastatin, or in combination, or sequentially with radiation, or at least one chemotherapeutic agent.

20. The compound according to any one of claims 1 to 10 or 12 to 18, for use in the treatment of a proliferative disorder or disease state, in combination with a death receptor agonist.

21. The compound according to any one of claims 1 to 10 or 12 to 18, for use in the treatment or prevention of arthritis, or immune hypersensitivity.

22. The compound according to any one of claims 1 to 10 or 12 to 18, for use in the treatment or prevention of autoimmune disease.

23. The compound according to any one of claims 1 to 10 or 12 to 18, for use in the treatment or prevention of inflammation, or infectious disease.

24. The compound according to any one of claims 1 to 10 or 12 to 18, for use in the prevention or treatment of thrombosis, heart attacks, or stroke.

25. The use of a compound according to any one of claims 1 to 10, for the preparation of a pharmaceutical composition for use in the treatment of a subject suffering from a protein kinase mediated disease, disorder, or condition in which Tec kinase family member activity is implicated.

26. The use of a compound according to any one of claims 1 to 10, for the preparation of a pharmaceutical composition for use in the treatment of a subject suffering from a protein kinase mediated disease, disorder, or condition in which Src kinase family member activity is implicated.

27. The use of a compound according to any one of claims 1 to 10, for the preparation of a pharmaceutical composition for use in the treatment of a subject suffering from a protein kinase mediated disease, disorder, or condition in which Btk kinase family member activity is implicated.

28. A pharmaceutical composition comprising a compound according to any one of claims 1 to 10 or a pharmaceutically acceptable salt, solvate, solvate of salt, stereoisomer, tautomer, isotope, prodrug, complex or biologically active metabolite thereof, and at least one pharmaceutically acceptable carrier, diluent, or excipient.

29. The pharmaceutical composition of claim 28, for use in a modulating kinase activity in a human or animal subject.

30. The pharmaceutical composition according to claim 28, for use in the treatment of a subject suffering from a protein kinase mediated disease, disorder, or condition in which Tyrosine kinase family member activity is implicated.

31. The pharmaceutical composition according to claim 28, wherein the pharmaceutical composition is for use in the treatment of a subject suffering from a protein kinase mediated disease, disorder, or condition associated with Src kinase family members.

32. The pharmaceutical composition according to claim 28, for use in the treatment of a subject suffering from a protein kinase mediated disease, disorder, or condition, wherein a protein kinase mediated disease, is associated with inhibiting a Btk kinase activity.

33. The pharmaceutical composition according to claim 28, for use alone or in combination with other agents in the treatment of a subject suffering from a protein kinase mediated disease, disorder, or condition in which Tyrosine kinase family member activity is implicated.

34. The pharmaceutical composition according to claim 28, for use alone or in combination with other agents in the treatment of a subject suffering from a protein kinase mediated disease, disorder, or condition in which Src kinase family member activity is implicated.

35. The pharmaceutical composition according to claim 28, for use alone or in combination with other agents in the treatment of a subject suffering from a protein kinase mediated disease, disorder, or condition in which Btk kinase family member activity is implicated.

36. The pharmaceutical composition according to any one of claims 28 to 35, for use in treatment or prevention of arthritis, or immune hypersensitivity.

37. The pharmaceutical composition according to any one of claims 28 to 35, for use in the treatment or prevention of autoimmune disease.

38. Use of a compound according to any one of claims 1 to 10 or a pharmaceutical composition according to claim 28, for treating or preventing disorder, or disease state characterized by inflammation, or cell proliferation.

39. Use of a compound according to any one of claims 1 to 10 or a pharmaceutical composition according to claim 28, for use in inhibiting protein kinase activity in a human or animal cell, or tissue.

40. A probe comprising a compound according to any one of claims 1 to 10 or a detectable label, or affinity tag for said compound.

41. The probe according to claim 40, wherein the detectable label is selected from the group consisting of: a fluorescent moiety, a chemiluminescent moiety, a paramagnetic contrast agent, a metal chelate, a radioactive isotope-containing moiety, or biotin.

42. An intermediate compound represented by:
Formula 2-c 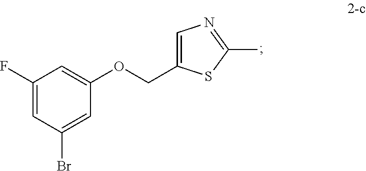
by Formula 3-b 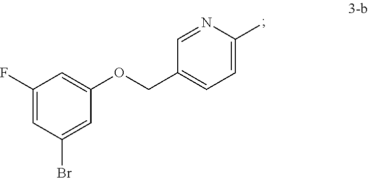
by Formula 4-b 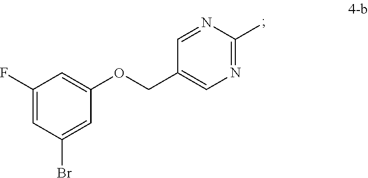
by Formula 5-b 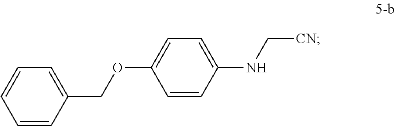 [SMILES: N#CCNc1ccc(OCc2ccccc2)cc1]
by Formula 6-b 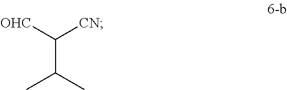
by Formula 7-c 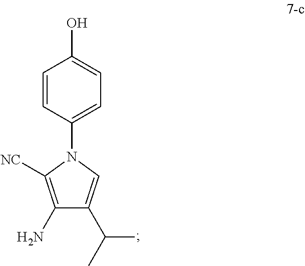
by Formula 8-d 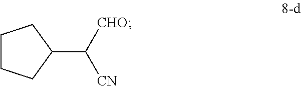
by Formula 9-c 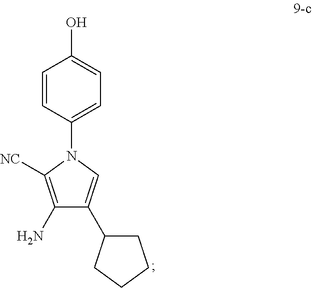 [SMILES: N#Cc1c(N)c(C2CCCC2)cn1-c1ccc(O)cc1]
by Formula 10-d 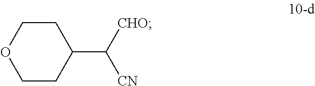 [SMILES: N#CC(C=O)C1CCOCC1]
by Formula 11-c 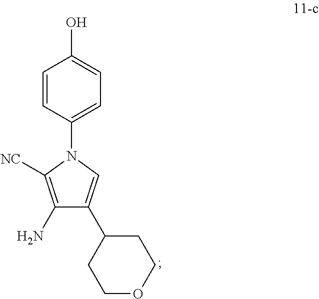 [SMILES: N#Cc1c(N)c(C2CCOCC2)cn1-c1ccc(O)cc1]
by Formula 21-g 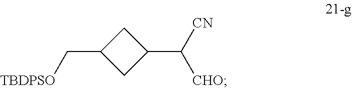
by Formula 22-c 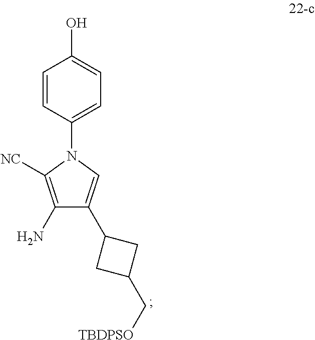
by Formula 24-d 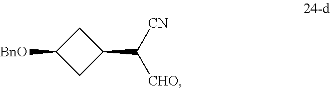 [SMILES: N#CC(C=O)[C@H]1C[C@@H](OCc2ccccc2)C1]
or
by Formula 25-c 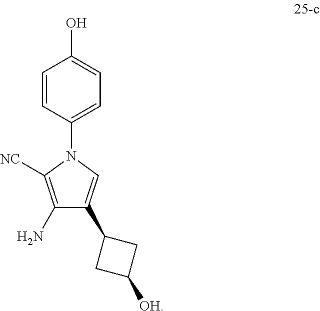 [SMILES: N#Cc1c(N)c([C@H]2C[C@@H](O)C2)cn1-c1ccc(O)cc1]
